(19) United States
(12) Patent Application Publication
(10) Pub. No.: US 2008/0299170 A1
(43) Pub. Date: Dec. 4, 2008

(54) Medical Devices and Coatings Therefor

(75) Inventors: Peter A. Lambert, Sutton Coldfield (GB); Martin Griffin, Bleasby (GB); Daniel Rathbone, Earlsdon (GB); Russell Collighan, Ilkeston (GB)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Aston University, Birmingham (GB)

(21) Appl. No.: 11/996,112

(22) PCT Filed: Jul. 12, 2006
(86) PCT No.: PCT/GB2006/002587

§ 371 Date: Jul. 14, 2008

(30) Foreign Application Priority Data
Jul. 21, 2005 (GB) 0515003.2

(51) Int. Cl. A61F 2/02; A61K 31/401; A61K 31/198; A61P 43/00

(52) U.S. Cl. 424/423; 514/428; 514/567

(57) ABSTRACT

A coating for a medical device, the device generally being of the type which is to be used internally in a subject, includes a transglutaminase (TGase) inhibitor. The Tgase inhibitor effectively prevents the activity of any Tgase involved in clot formation or clot stability. Preferably, the transglutaminase inhibitor is a factor XIIIa inhibitor. The coating may be prepared by suitable processes such as esterification. The coating may be immobilized on a suitable medical device in a number of ways and is preferably included in a TGase inhibitor containing polymer. The coating may also include a suitable antimicrobial agent.

### FIELD OF THE INVENTION

The present invention relates to the field of medical devices and to solving problems associated with their use inside patients. More particularly, the present invention relates to transglutaminase inhibitors, particularly factor XIIIa inhibitors and their inclusion in coatings for medical devices. The medical devices coated with coatings according to invention provide a number of significant clinical benefits for subjects as detailed herein.

### BACKGROUND TO THE INVENTION

A number of medical devices, which require introduction into a subject, are routinely used in the clinic. There are numerous disadvantages for a subject which result from the introduction of these devices into their bodies. One primary example of such a device is the central venous catheter (CVC), and problems associated with CVC use, which apply equally to other medical devices for internal use (because they incite the same or a similar response) are described below.

CVCs are used with increasing frequency in intensive care, general medicine and oncology to provide venous access for repeated drug administration, measurement of central venous pressure and withdrawal of blood samples. The major complications of CVCs (and other medical devices for internal use) are thrombotic occlusion and infection by skin organisms, principally staphylococci. Despite routine flushing with saline or heparin, approximately 40% of CVCs result in thrombosis of the blood vessel and this greatly increases the risk of infection. Most CVCs become coated with a fibrin sheath within days of insertion and CVC-associated thrombi arise within 30 days, causing post-phlebitic syndrome or pulmonary embolism[1].

Restoration of CVC function (patency) requires fibrinolytic treatment to remove thrombotic occlusions whilst treatment of infection is problematic, necessitating use of antibiotic locks or catheter removal[2]. In cancer patients, systemic prophylactic anti-coagulant therapy with both low-molecular weight heparin and low-dose warfarin significantly reduced the rate of catheter-related thrombosis[3]. Newer anti-coagulant treatments involving heparin pentasaccharide (e.g. fondaparinux[4]) and direct thrombin peptide inhibitors (e.g. melagatran/ximelagatran[5]) are now under evaluation for prevention of thrombo-embolism in at-risk patient groups.

Thus, at present, the problems associated with medical devices introduced internally into a subject have not been addressed in a satisfactory manner. There is a need for medical devices which can be introduced into a subject's body and which can remain in the body without the requirement for either flushing or removal for cleaning, or other treatments to prevent thrombus development and (bacterial) infections.

Transglutaminases (TGases) catalyse the calcium-dependent cross-linking between proteins through formation of a peptidase-stable isopeptide linkage between glutamine and lysine residues on separate protein molecules[6]. The factor XIIIa (FXIIIa) component involved in blood coagulation is a TGase that catalyzes the covalent cross-linking of fibrin, affording the clot additional structural and mechanical stability and resistance to plasmin-mediated degradation via the incorporation of plasminogen activator inhibitors[7]. A further transglutaminase which has also been implicated in blood clotting is found inside the red blood cells. The role of this enzyme in clot stabilisation has yet to be established but potential exists for therapeutic applications via its inhibition [7a].

Bacteria can bind to the host proteins, fibrin/fibrinogen and fibronectin. The interaction is mediated by the production of a number of microbial surface components recognizing adhesive matrix molecules. In Staphylococcus aureus these include the fibrinogen-binding clumping factors A and B and the fibronectin-binding protein (FnbA) [8]. FnbA is a substrate for FXIIIa and undergoes covalent cross-linking to fibrinogen and fibronectin[9,10]. Other organisms commonly associated with catheter-related infection, particularly, Staphylococcus epidermidis and the yeast, Candida albicans also produce fibronectin binding surface proteins [10a,b].

Various TGase inhibitors are known in the art. These include competitive amine inhibitors, competitive glutamine inhibitors and irreversible inhibitors. Competitive amine inhibitors include dansylcadaverines [24, 25] and N-phenyl-N′-(ω-aminoalkyl) thioureas [26]. Competitive glutamine inhibitors include aliphatic amides [27], dipeptides [28] and polypeptides [29]. Irreversible inhibitors include iodoacetamide [28, 30], phenol-containing halomethyl ketones [31], alkylisocyanates [32], α-halomethylcarbonyl inhibitors [33], dihydroisoazoles (U.S. Pat. No. 4,912,120), azoles, azolium salts (U.S. Pat. No. 4,968,713), thiadiazoles [34] and epoxides [35].

WO 2004/113363 (The Nottingham Trent University) discloses a number of TGase inhibitors of general Formula I and is incorporated herein by reference.

U.S. Pat. No. 5,098,707 (Merck) describes imidazolium salts and their inclusion in compositions for inhibiting clot formation. The compounds are purported to inhibit TGase activity. The compounds disclosed in U.S. Pat. No. 5,098,707 have utility in the present invention. The disclosure of U.S. Pat. No. 5,098,707 is incorporated herein by reference.

WO 2005/049064, incorporated herein by reference, discloses transglutaminase inhibitors and their use in treatment of Celiac sprue.

EP0237082, which is hereby incorporated by reference, also discloses transglutaminase inhibitors and their use in treatment of acne.

### DESCRIPTION OF THE INVENTION

The present invention is based around an inventive insight into the link between medical device-associated thrombus formation and infections by organisms such as those usually found on the skin of a subject (such as staphylococci for example) which can cause infection when a medical device (such as a catheter for example) is inserted into a subject.

This link is related to the mechanism by which micro-organisms, including bacteria and yeast such as staphylococci and candida, colonise surfaces of medical devices leading to infection. These organisms can bind to the host proteins, fibrin/fibrinogen and fibronectin, particularly when such proteins are deposited upon the surface of medical devices. The interaction is mediated by the production of a number of microbial surface components recognizing adhesive matrix molecules. In Staphylococcus aureus these include the fibrinogen-binding clumping factors A and B and the fibronectin-binding protein (FnbA) [8]. FnbA is a substrate for factor XIIIa and undergoes covalent cross-linking to fibrinogen and fibronectin[9,10]. Consequently S. aureus becomes covalently cross-linked to fibronectin and fibrin during deposition within the fibrin-platelet matrix of thrombi on the surface of medical devices, preventing release into the blood during natural fibrinolysis and retaining the organisms in an environment protected from antibiotic action and host defences. Staphylococcus epidermidis and Candida albicans may also become covalently attached by similar crosslinkage via their fibronectin receptor proteins.

Thus, the inventors have surprisingly discovered that incorporation of TGase inhibitors onto the surfaces of medical devices has multiple beneficial effects due to their ability to disrupt clot formation and/or stabilisation. Firstly, a protected environment for micro-organisms, such as bacteria and yeast away from antimicrobial agent action and host defences is prevented by disrupting covalent attachment of micro-organisms, such as bacteria and yeast to host proteins. Release of micro-organisms, such as bacteria and yeasts during thrombolysis renders them susceptible to therapy using appropriate antimicrobial agents and/or to killing by the subject's immune system. Secondly, the incorporation of TGase inhibitors also increases the rate of thrombolysis at the surface of the medical device by destabilising any forming clots by inhibiting fibrin cross-linking. Furthermore, by targeting this step in the clotting process, the incorporation of plasminogen activator inhibitors into the clot is prevented. This effectively makes the thrombus, which cannot fully form and stabilise due to the activity of the TGase inhibitor, more susceptible to degradation/lysis.

### Coatings of the Invention

Accordingly, in a first aspect, the invention provides a coating for a medical device, which device is for introduction into a subject, comprising, consisting essentially of or consisting of a transglutaminase (TGase) inhibitor. The TGase inhibitor prevents and/or affects and/or reduces the activity of any TGase involved in clot formation and/or stability. Preferably, the transglutaminase inhibitor comprises, consists essentially of or consists of a factor XIIIa inhibitor. However, inhibition of other TGases may also prove beneficial according to the invention provided the TGases are involved in clot formation and/or stability. For example, inhibition of the TGase which is found in red blood cells and which is implicated in blood clotting may also be beneficial [7a].

According to all aspects of the invention, the subject is preferably a human subject. The human will preferably be in need of some form of treatment, which involves the introduction of a medical device into the subject.

By “transglutaminase inhibitor” is meant, according to all aspects of the present invention, any substance that can act to prevent and/or affect and/or decrease the transglutaminase activity of the TGase, which is preferably factor XIIIa. Thus, the inhibitor must be capable of inhibiting the ability of any transglutaminase which may be involved in fibrin crosslinking to catalyze covalent cross linking of fibrin and/or incorporation of plasminogen activator inhibitors into a forming clot. For all aspects of the invention it is to be understood that a single transglutaminase inhibitor may be utilised or a combination of multiple inhibitors may be utilised to coat the same medical device. As aforementioned, preferably at least factor XIIIa activity is inhibited. Preferably, the TGase inhibitor is a small molecule but may comprise a biological agent, such as an antibody or derivative or fragment thereof which retains binding affinity, for example.

A preferred class of TGase inhibitors is the so-called “suicide inhibitors”. These inhibitors irreversibly inhibit the activity of the TGase and can be contrasted with competitive substrates. For example, they may alter the structure of the TGase active site so that it no longer acts on its substrate.

Exemplary TGase inhibitors, not intended to be limiting on the scope of the invention, comprise, consist essentially of or consist of any one or more of:
(a) Competitive amine inhibitors including dansylcadaverines [24, 25] and N-phenyl-N′-(ω-aminoalkyl) thioureas [26],
(b) Competitive glutamine inhibitors including aliphatic amides [27], dipeptides [28] and polypeptides [29],
(c) Irreversible inhibitors including iodoacetamide [28, 30], phenol-containing halomethyl ketones [31], alkylisocyanates [32], α-halomethylcarbonyl inhibitors [33], dihydroisoazoles (U.S. Pat. No. 4,912,120), azoles, azolium salts (U.S. Pat. No. 4,968,713), thiadiazoles [34] and epoxides [35],
(d) the TGase inhibitors described in WO 2004/113363 (incorporated herein by reference) in particular the specific examples labelled (a) to (s) therein;
(e) imidazolium salts as disclosed in U.S. Pat. No. 5,098,707 (incorporated herein by reference);
(f) TGase inhibitors as disclosed in EP0237082, which is hereby incorporated by reference; and
(g) the TGase inhibitors described in WO 2005/049064 (incorporated herein by reference), which comprise derivatives of 3-halo-4,5-dihydroisoxazole.

With respect to (d) above, the TGase inhibitors which are useful in the present invention may comprise, consist essentially of or consist of TGase inhibitors of the general formula I:

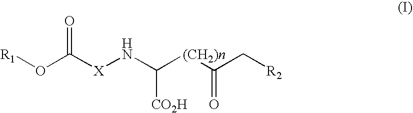

wherein:
X represents an amino acid group;
n is an integer between 1 and 4;
R₁ represents benzyl, t-butyl or 9-fluorenylmethyl; and
R₂ represents

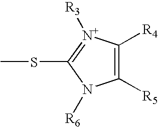

wherein R₃, R₄, R₅ and R₆ each independently represent lower alkyl or —S⁺R₇R₈ wherein R₇ and R₈ each independently represent lower alkyl or a pharmaceutically and/or veterinarily acceptable derivative thereof. Such derivatives include salts and solvates, such as acid or base addition salts. Pharmaceutically and/or veterinarily acceptable counter-anions may also be utilised, such as halides, in particular bromide counter-anions.

Advantageously, X is an L-amino acid group.

Preferably, X is selected from the group consisting of phenylalanine, glutamine (including N-substituted derivatives thereof, such as N-substituted piperidinyl and propyl derivatives), isoleucine, alanine, glycine, tyrosine, proline, serine, lysine and glutamic acid. Thus, preferred compounds for use in the invention include N-benzyloxycarbonyl-L-glutamyl-y-isopropylamide-6-dimethyl-sulfonium-5-oxo-L-norleucine bromide salt and N-benzyloxycarbonyl-L-glutamyl-Y-piperidinamide-6-dimethylsulfonium-5-oxo-L-norleucine bromide salt.

In one embodiment, ‘n’ is 2. Advantageously, ‘R₁’ is benzyl.

Conveniently, ‘R₂’ represents

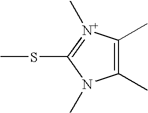

Preferably, ‘R₂’ represents —S⁺R₇R₈, wherein R₇ and R₈ each independently represent lower alkyl.

The term “lower alkyl” is intended to include linear or branched, cyclic or acyclic, C₁-C₅ alkyl, which may be saturated or unsaturated. Lower alkyl groups which R₃, R₄, R₅, R₆, R₇ and/or R₈ may represent include C₁-C₄ alkyl, C₁-C₃ alkyl, C₁-C₂ alkyl, C₂-C₅ alkyl, C₃-C₅ alkyl, C₄-C₅ alkyl, C₂-C₄ alkyl, C₂-C₃ alkyl and C₃-C₄ alkyl. Preferred lower alkyl groups which R₃, R₄, R₅, R₆, R₇ and/or R₈ may represent include C₁, C₂, C₃, C₄ and C₅ alkyl.

Preferably, R₃, R₄, R₅, R₆, R₇ and/or R₈ are —CH₃ or —CHCH₂. More preferably, R₃, R₄, R₅, R₆, R₇ and/or R₈ are —CH₃.

Preferred compounds are selected from the group consisting of (the structural formulae for these compounds are listed under the same heading in WO 2004/113363, which disclosure is incorporated herein by reference):
(a) N-Benzyloxycarbonyl-L-phenylalanyl-6-dimethylsulfonium-5-oxo-L-norleucine
(b) N-Benzyloxycarbonyl-L-glutaminyl-6-dimethylsulfonium-5-oxo-L-norleucine
(c) N-Benzyloxycarbonyl-L-isoleucinal-6-dimethylsulfonium-5-oxo-L-norleucine
(d) N-Benzyloxycarbonyl-L-phenylalanyl-7-dimethyl-sulfonium-6-oxo-heptanoic acid
(e) N-Benzyloxycarbonyl-L-phenylalanyl-L-5-dimethylsulfonium-4-oxo-norvaline
(f) N-Benzyloxycarbonyl-L-alaninal-6-dimethylsulfonium-5-oxo-L-norleucine
(g) N-Benzyloxycarbonyl-L-glycinal-6-dimethylsulfonium-5-oxo-L-norleucine
(h) N-Benzyloxycarbonyl-L-tyrosinal-6-dimethylsulfonium-5-oxo-L-norleucine
(i) N-Benzyloxycarbonyl-L-prolinyl-6-dimethylsulfonium-5-oxo-L-norleucine
(j) N-Benzyloxycarbonyl-L-serinyl-6-dimethylsulfonium-5-oxo-L-norleucine
(k) N-Benzyloxycarbonyl-L-glutaminyl-6-dimethylsulfonium-5-oxo-L-norleucine
(l) N-α-Benzyloxycarbonyl-N-ε-trifluoroacetate-L-lysinyl-6-dimethylsulfonium-5-oxo-L-norleucine
(m) N-α-Benzyloxycarbinyl-γ-piperidinyl-6-dimethylsulfonium-5-oxo-L-noreucine
(n) N-a-Benzyloxycarbonyl-γ-propyl-L-glutaminyl-6-dimethylsulfonium-5-oxo-L-norleucine
(o) N-Benzyloxycarbonyl-L-phenylalanyl-6-diethylsulfonium-5-oxo-L-norleucine
(p) N-α-Benzyloxycarbonyl-L-phenylalanyl-6-tetra-methylmercaptoimidazole-5-oxo-L-norleucine
(q) N-9-Fluorenylmethyloxycarbonyl-L-phenylalanyl-6-dimethylsulfonium-5-oxo-L-norleucine
(r) N-α-tert-butyloxycarbonyl-L-phenylalanyl-6-dimethylsulfonium-5-oxo-L-norleucine
(s) N-Benzyloxycarbonyl-L-prolinyl-6-tetra-methylmercaptoimidazole-5-oxo-L-norleucine

It will be appreciated by persons skilled in the art that pharmaceutically, and/or veterinarily, acceptable derivatives of the compounds of formula I, such as salts and solvates, are also included within the scope of the invention. Salts which may be mentioned include: acid addition salts, for example, salts formed with inorganic acids such as hydrochloric, hydrobromic, sulfuric and phosphoric acid, with carboxylic acids or with organo-sulfonic acids; base addition salts; metal salts formed with bases, for example, the sodium and potassium salts.

Thus, the compounds of formula I may be counterbalanced by counteranions. Exemplary counter-anions include, but are not limited to, halides (e.g. fluoride, chloride and bromide), sulfates (e.g. decylsulfate), nitrates, perchlorates, sulfonates (e.g. methane-sulfonate) and trifluoroacetate. Other suitable counter-anions will be well known to persons skilled in the art.

Preferably, the compound is a bromide salt.

It will be further appreciated by skilled persons that the compounds of formula I may exhibit tautomerism. All tautomeric forms and mixtures thereof may be utilised within the scope of the invention.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

Preferably, the compounds comprise L amino acid groups.

For the avoidance of doubt, it is hereby stated that all stereoisomers of all compounds described herein are intended to fall within the scope of the invention, provided they act as inhibitors of transglutaminase mediated fibrin stabilisation and/or inhibitors of the crosslinking of a pathogenic microorganism to the host/subject proteins that form a constituent part of a thrombus.

In a preferred embodiment, the inhibitor comprises, consists essentially of or consists of a TGase inhibitor comprising, consisting essentially of or consisting of the following structure; represented as formula II:

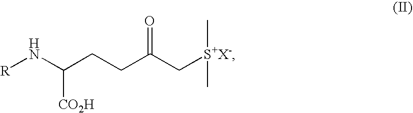

preferably the stereoisomeric form shown below;

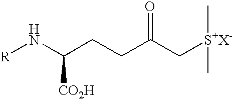

wherein R=a phenylalanine or proline substituent
X=a pharmaceutically and/or veterinarily acceptable counter anion, in particular a halide counter anion and preferably bromide.

By phenylalanine or proline substituent is meant any molecule/group which includes the essential structural elements of the amino acid phenylalanine or the amino acid proline and includes the amino acids phenylalanine and proline and derivatized and substituted versions thereof.

The substituent is designed to mimic a peptide chain and may include further substituents in addition to the basic phenylalanine and proline groups. Preferred additional substituents include the following group:

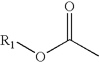

wherein R₁ represents benzyl, t-butyl or 9-fluorenylmethyl. Particularly preferred inhibitors comprise, consist essentially of or consist of TGase inhibitors which comprise, consist essentially of or consist of the formulas/structures:

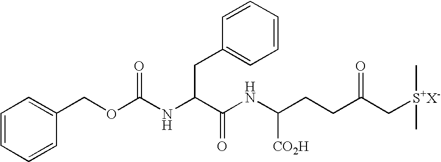

N-benzyloxycarbonyl-L-phenylalanyl-6-dimethylsulfonium-5-oxo-L-norleucine;
   preferably, the stereoisomer:

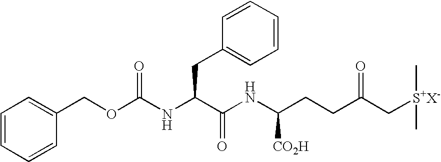

referred to hereinafter as compound 1 and;

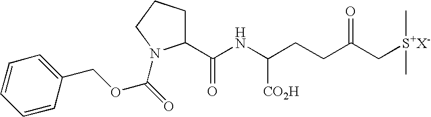

N-Benzyloxycarbonyl-L-prolinyl-6-dimethylsulfonium-5-oxo-L-norleucine;
   preferably, the stereoisomer:

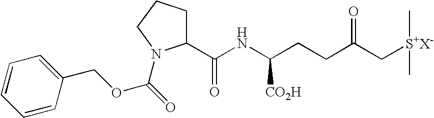

referred to hereinafter as compound 2;
wherein X=a pharmaceutically and/or veterinarily acceptable counter anion, in particular a halide counter anion and preferably bromide (i.e. as defined above)

With respect to (e) above, the imidazole compounds for use in the present invention may be selected from the group consisting of:

(A) an imidazole represented by the formula (III);

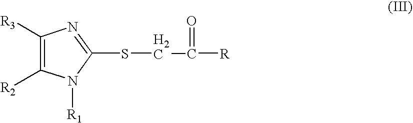

or its acid addition salt, and
(B) an imidazolium salt represented by the formula (IV);

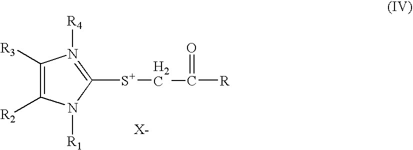

In the above and subsequent formulas:

R is hydrogen; lower alkyl; substituted lower alkyl wherein the substituents are selected from hydroxy, lower alkoxy, phenoxy, phenylthio, 2-pyridinyl-N-oxide-thio, and halo; cycloalkyl from 3 to 6 carbon atoms; benzyl; substituted benzyl wherein the substituents are selected from halo, hydroxy, lower alkyl and lower alkoxy; phenyl; substituted phenyl containing 1 to 3 substituents selected from hydroxy, lower alkoxy, carbo(lower alkoxy), carbamido, N-(lower alkyl)carbamido or cyano; pyridyl pyrimidinyl; or pyrazinyl; lower alkyl; substituted lower alkyl wherein the substituent is carbalkoxy or carbamido; or ArCₙH²ₙ— wherein Ar is phenyl, (lower alkyl)phenyl, (lower alkoxy)phenyl, or halophenyl and n is 1-3; R₂ is nitro; carbo(lower alkoxy); halo; cyano; phenyl; substituted phenyl containing from 1 to 3 substituents selected from lower alkyl, lower alkoxy, halo, and hydroxy; phenoxy; phenylthio; an amido group represented by —NHCOQ wherein Q is lower alkyl, —CH(NH₂)CH₂C₆H₅ or —NH(lower alkyl); a hydroxyalkyl group represented by

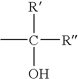

wherein R′ is hydrogen or R″ wherein R″ is hydrogen, phenyl, phenoxyphenyl, biphenylyl, (lower alkyl)phenyl, lower alkyl and lower cycloalkyl, or R′ and R″ taken together is alkylene from 4 to 6 carbon atoms; or an ether-alkyl group represented by

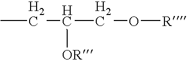

wherein R′″ is hydrogen, —CO-(lower alkyl), —CO-phenyl, —CO-biphenylyl, —CO-phenyl-O-phenyl and —CONH-phenyl, and R″″ is phenyl or lower alkyl; R₃ is hydrogen, or when R₂ is phenyl or substituted phenyl is optionally the same as R₂ or R₂ and R₃ taken together may be alkylene from 3 to 10 carbon atoms optionally substituted with phenyl or spiroalkylene, or benzo; R₄ is lower alkyl, ArCnH₂n wherein Ar is phenyl, (lower alkyl)phenyl, (lower alkoxy)phenyl, or halophenyl, and n is 1-3; and X is an anion of a pharmaceutically acceptable salt.

By the expressions “lower alkyl” and “lower alkoxy” as employed in the specification and claims are meant radicals having from 1 to 6 carbon atoms.

By the expression “spiroalkylene” is meant an alkylene chain of from 3 to 6 carbon atoms, the end carbons of which are attached to the same carbon of the nucleus.

By the expression “halo” is meant fluoro, chloro, bromo and iodo.

Pharmaceutically acceptable salts suitable as acid addition salts as well as providing the anion of the imidazolium salts are those from acids such as hydrochloric, hydrobromic, hydroiodic, phosphoric, sulfuric, trifluoroacetic, trichloroacetic, oxalic, maleic, pyruvic, malonic, succinic, citric, mandelic, benzoic, cinnamic, methanesulfonic, ethanesulfonic, trifluoromethanesulfonic and the like, and include other acids related to the pharmaceutically acceptable salts listed in Journal of Pharmaceutical Science, 66, 2 (1977) and incorporated herein by reference.

The compounds, both those which are acid addition salts of the compounds represented by formula (III) and those quaternary salts represented by formula (IV) are solids soluble in polar solvents such as water, methanol, ethanol and the like. The imidazoles of formula (III) are soluble in non-polar solvents such as ethyl acetate, methylene chloride, ethylene dichloride, carbon tetrachloride, and the like.

A specific compound which may be employed in the present invention is one comprising, consisting essentially of or consisting of the formula V:

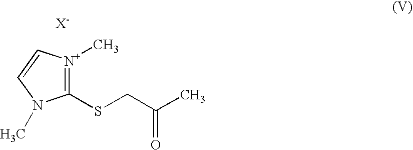

wherein X is as defined above (a pharmaceutically and/or veterinarily acceptable counter anion, in particular a halide counter anion and preferably bromide).

With respect to (f) above, inhibitors of the following general formula (VI), as disclosed in EP0237082 (which reference is incorporated herein in its entirety), may also be utilised in the present invention:

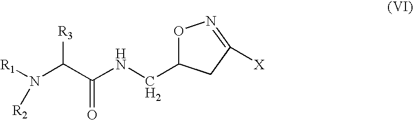

or an optical isomer thereof, or a pharmaceutically acceptable salt thereof, wherein:
R₁ and R₂, together with the nitrogen atom to which they are attached, together represent phthalimido; or R₁ and R₃ together form —CH₂—CH₂—CH₂— or CH₂—CHOH—CH₂; or R₁, R₂ and R₃ are defined as follows:
R₁ is hydrogen or methyl;
R₂ is selected from the group consisting of:
(1) hydrogen;
(2) alkyl;
(3) lower alkyl sulfonyl;
(4) aryl sulfonyl;
(5) aryl sulfonyl substituted with lower alkyl on the aryl moiety;
(6) 9-fluorenylmethyloxycarbonyl, succinyl or cinnamoyl;
(7) a radical of the formula (VII):

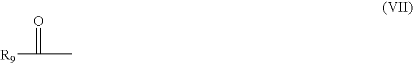

wherein:
R₉ is hydrogen; alkyl of 1 to 4 carbon atoms; aryl; aryl substituted with up to 2 substituents where the substituents are independently halo, lower alkyl, alkoxy, nitro, trifluoromethyl, carboxyl, or alkoxycarbonyl; aralkyl; pyridinyl; furanyl; alkoxy; aralkoxy; aralkoxy substituted on the aryl radical with up to 2 substituents where the substituents are independently halo, lower alkyl, alkoxy, nitro, or trifluoromethyl; adamantyloxy; aralkylamino; or aralkyl substituted on the aryl radical with up to 2 substituents where the substituents are independently hydroxy, alkoxy or halo; and
(8) a radical of the formula (VIII)

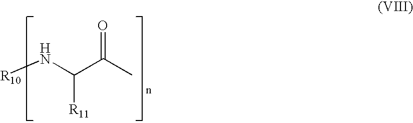

wherein: n=0 or 1;
R₁₀ is independently hydrogen, alkyl or the radical defined by formula (V) above; R₁₁ is selected from the group consisting of hydrogen; lower alkyl; —(CHR₁₂)ₘWR₁₃
wherein m is 1 or 2, W is oxygen or sulfur and R₁₂ and R₁₃ are independently hydrogen or methyl; —CH(CH₃)—OCH₂C₆H₅; —(CH₂)ₖC(O)Y wherein k is 1 or 2 and Y is hydroxyl, amino, alkoxy, or aralkoxy; (CH₂)pNHCH(NHR₁₄)NR₁₅ wherein p is 2, 3, or 4 and R₁₄ and R₁₅ are independently hydrogen or lower alkyl; (CH₂)_{q}NH₂ wherein q is 2, 3, 4, or 5; —(CH₂)₄NHCOOC(CH₃)₃; —(CH₂)₂CHOHCH₂NH₂; a radical of formula (IX)

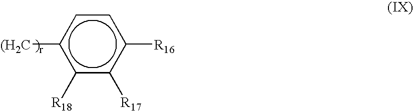

wherein r is 1 or 2 and R₁₆, R₁₇ and R₁₈ are independently hydrogen, hydroxyl, halo, methoxy, lower alkyl, halo lower alkyl, amino, N-protected amino, guanidino, nitro, cyano, —COOH, —CONH2, —COOR′″ where R′″ is lower alkyl or —OR* where R* is an O-protecting group; and a radical chosen from

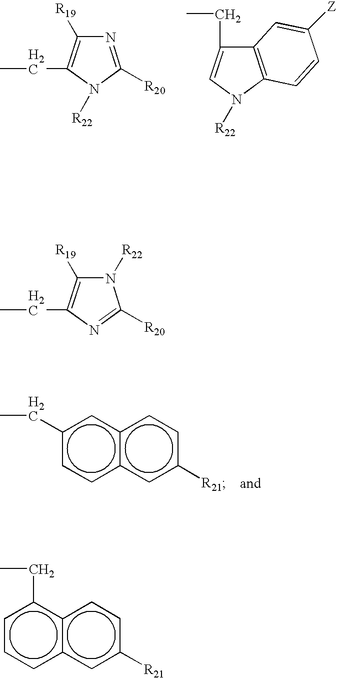

wherein R₁₉ and R₂₀ are independently hydrogen, lower alkyl, halo or trifluoromethyl alkyl; R₂₁ is hydrogen, hydroxy or methoxy; and Z is hydrogen, hydroxyl, or —OR* where R* is an O-protecting group; R₂₂ is hydrogen or an N-protecting group for imidazole or indole functionalities; R₃ is independently selected from the group recited for R₁₁ above; X is selected from the group consisting of: halo; —OR, —SR, —S(O)R, —S(O)₂R, —S(O)₂NH₂ or —S(O)₂NHR wherein R is lower alkyl mono-, di- or tri-fluoro alkyl of 2 or 3 carbon atoms, aryl, or optionally substituted aryl; —NR′R″ wherein R′ and R″ are independently hydrogen, lower alkyl, or aryl; and

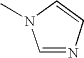

For compounds under heading (f) the definitions of EP 0237082 apply. Thus; “Alkyl” means a branched or unbranched, saturated aliphatic hydrocarbon radical, having the number of carbon atoms specified, or if no number is specified, having up to 8 carbon atoms. The prefix “alk-” is also indicative of a radical having up to 8 carbon atoms in the alkyl portion of that radical, unless otherwise specified. Examples of alkyl radicals include methyl, ethyl, n-propyl, isopropyl, n-butyl, tert-butyl and the like. The terms “lower alkyl” and “alkyl of 1 to 4 carbon atoms” are synonymous and used interchangeably.

“Alkoxy” means an alkyl radical of up to 8 carbon atoms unless otherwise specified, that is attached to an oxygen radical, which is in turn attached to the structure provided. Examples are, methoxy, ethoxy, propoxy, n-butoxy, isobutoxy, tert-butoxy, n-pentoxy, n-hexoxy, n-heptoxy, n-octoxy, and the like.

“Alkoxycarbonyl” means an alkoxy radical (as defined above) attached to a carbonyl radical, which in turn is attached to the structure provided. Examples are methoxycarbonyl, ethoxycarbonyl, propoxycarbonyl, n-butoxycarbonyl, isobutoxycarbonyl, tert-butoxycarbonyl, n-pentoxycarbonyl, n-hexoxycarbonyl, n-heptoxycarbonyl, n-octoxyearoonyl, and the like.

“Aralkoxy” means an aralkyl radical (as defined below) that is attached to an oxygen radical, which is in turn attached to the structure provided. Examples are benzyloxy, naphthylmethoxy, and the like.

“Aralkyl” means an aryl group (as defined below) attached to a lower alkyl radical, which is in turn attached to the structure provided. Examples are, benzyl, naphthylmethyl, and the like.

“Aryl” means phenyl, 1-naphthyl or 2-naphthyl.

“Boc” means t-butyloxycarbonyl.

“BOC-ON” is C2-(tertbutoxycarbonyloxyimino)-2-phenylacetylnitrile].

“Cbz” means benzyloxycarbonyl.

“DCC” means N,N′-dicyclohexylcarbodiimide.

“DMAP” means 4-dimethylaminopyridine.

“EDCI” means l-(3-dimethylaminopropyl)-3-ethylcarbodiimide.

“Fmoc” means 9-fluorenylmethyloxycarbonyl.

“Halo” means bromo, chloro, fluoro or iodo.

“N-Protecting groups” can be considered to fall within five classes: N-acyl, N-alkoxycarbonyl, N-arylmethoxycarbonyl, N-arylmethyl, and N-arylsulfonyl protecting groups. An N-acyl protecting group is a lower alkyl carbonyl radical, a trifluoroacetyl radical. An N-alkoxycarbonyl protecting group is a lower alkoxycarbonyl radical. An N-arylmethoxycarbonyl protecting group is a 9-fluoroenemethoxycarbonyl radical (Fmoc); or benzyloxycarbonyl radical which can optionally be substituted on the aromatic ring with methoxy, nitro, chloro, or o-chloro. An N-arylmethyl protecting group is a benzyl radical, which can optionally be substituted on the aromatic ring with methoxy, nitro, orchloro. An N-arylsulfonyl protecting group is a phenylsulfonyl radical, which can optionally be substituted on the aromatic ring with methyl (“tosyl”) or methoxy.

“N-Protecting groups” for imidazole functionalities on histidine amino acid side chains are known in the art, and described in “The Peptides,” Vol. 3, pp. 70-80, and “Chemistry of the Amino Acids”, Vol. 2, pp. 1060-1068. These include the benzyl, triphenylmethyl (trityl), 2,4-dinitrophenyl, p-toluenesulfonyl, benzoyl, and Cbz N-protecting groups.

“N-Protecting groups” for indole functionalities on tryptophan amino acid side chains are known in the art and described in “The Peptides,” Vol. 3, pp. 82-84. These include the formyl and Cbz N-protecting groups.

“O-Protecting groups” for hydroxy functionalities on amino acid side chains are known in the art and described in “The Peptides,” Vol. 3, pp. 169-201, and “Chemistry of the Amino Acids,” Vol. 2, pp. 1050-1056. For aromatic hydroxy functionalities, suitable O-protecting groups include the benzyl, acetyl, tert-butyl, methyl, Cbz, and tosyl groups.

“N-Protecting groups” for amine functionalities are well known in the art, and include Boc, Cbz, Fmoc, phthaloyl, benzoyl, mesyl, tosyl, and the like.

“Optional” or “optionally” means that the subsequently described event or circumstance may or may not occur, and that the description includes instances where said event or circumstance occurs and instances in which it does not. For example, “optionally substituted phenyl” means that the phenyl may or may not be substituted and that the description includes both unsubstituted phenyl and phenyl wherein there is substitution.

“Optionally substituted aryl” means aryl, aryl containing 1 to 5 fluoro substituents; or aryl containing 1 to 3 substituents, where the substituents are independently selected from the group consisting of alkoxy, alkyl, nitro, trifluoromethyl, —COOH, —COOR wherein R is lower alkyl or —CON₂H.

“Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Phthalimido” with respect to the compounds under heading (f) means that R₁, R₂ and the nitrogen to which they are attached together form the structure:

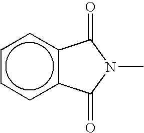

Preferred embodiments compounds of Formula (VI) wherein R₁ is hydrogen; R₂ is a radical of Formula (VII) as set forth above, wherein R₉ is selected from: pyridinyl; aryl; aryl substituted with up to 2 substituents where the substituents are independently halo, lower alkyl, alkoxy, nitro, trifluoromethyl, carboxyl or alkoxycarbonyl; aralkyl; alkoxy; aralkoxy; and aralkoxy substituted on the aryl radical with up to 2 substituents independently selected from halo, lower alkyl, alkoxy, nitro, and trifluoromethyl; adamantyloxy, aralkylamino, aralkyl substituted on the aryl radical with up to 2 substituents where the substituents are independently hydroxy, alkoxy, or halo; or R₂ is a radical of Formula (VIII) as set forth above, wherein R₁₀ is commensurate with the scope of Formula (VII) as set forth above in this paragraph; or R₁ and R₂ together with the nitrogen to which they are attached, represent phthalimido; and X is halo, —OR, —SR, —S(O)R, —S(O)₂R, —S(O)₂NH₂, or —S(O)₂NHR wherein R is aryl or optionally substituted aryl,

More preferred are those preferred compounds as defined in the immediately preceding paragraph, but wherein R₉ is alkoxy; aralkoxy; aralkoxy substituted on the aryl radical with up to 2 substituents independently selected from halo, lower alkyl, alkoxy, nitro, and trifluoromethyl; adamantyloxy, aralkylamino, aralkyl substituted on the aryl radical with up to 2 substituents where the substituents are independently hydroxy, alkoxy, or halo; and wherein R₁₀ is commensurate with the scope of Formula (VII) as defined in this paragraph (in accordance with the more preferred definition of R₉ as set forth in this sentence); and X is halo.

Most preferred are those more preferred compounds as defined in the immediately preceding paragraph, but R₉ is aralkoxy; adamantyloxy, aralkyl substituted on the aryl radical with up to 2 substituents where the substituents are independently hydroxy, alkoxy, orhalo; and wherein R₁₀ is commensurate with the scope of Formula (VII) as defined in this paragraph (in accordance with the most preferred definition of R₉ as set forth in this sentence); and X is chloro or bromo.

Specific examples of compounds for use in the present invention are:
5-(N-benzyloxycarbonyl-L-phenylalaninamidomethyl)-3-chloro-4,5-dihydroisoxazole;
5-(N-benzyloxycarbonyl-L-phenylalaninamidomethyl)-3-bromo-4,5-dihydroisoxazole; 5-(N-benzyloxycarbonyl-L-para-tyrosinamidomethyl)-3-bromo-4,5-dihydroisoxazole;
5-(N-benzyloxycarbonyl-L-ortho-tyrosinamidomethyl)-3-bromo-4,5-dihydroisoxazole;
5-(N-benzyloxycarbonyl-D-naphthylalaninamidomethyl)-3-chloro-4,5-dihydroisoxazole;
5-(N-benzyloxycarbonyl-D-para-chlorophenylalanin-amidomethyl)-3-chloro-4,5-dihydroisoxazole;
5-(N-tert-butoxycarbonyl-L-phenylalaninamidomethyl)-3-bromo-4,5-dihydroisoxazole;
5-(N-benzyloxycarbonyl-L-aspartic acid-aamidomethyl)-3-bromo-4,5-dihydroisoxazole;
5-(N-benzyloxyzarbonyl-L-glutamic acid-a amidomethyl)-3-bromo-4,5-dihydroisoxazole;
5-(N-benzyloxycarbonyl-N-e-tert-butoxycarbonyl-L-lysine-amidomethyl)-3-bromo-4,5-dihydroisoxazole;
5-(N-benzyloxycarbonyl-ss-benzyl-L-aspartic acid amidomethyl)-3-bromo-4,5-dihydroisoxazole;
5-(N-acetyl-L-naphthylalaninamidomethyl)-3-bromo4,5-dihydroisoxazole;
5-(N-benzyloxycarbonyl-glycinamidomethyl)-3-bromo4,5-dihydroisoxazole; 5-(N-benzyloxycarbonyl-L-isoleucinamidomethyl)-3-bromo-4,5-dihydroisoxazole;
5-(N-E 9-fluorenylmethyloxycarbonyl]-L-phenylalanin-amidomethyl)-3-bromo-4,5-dihydroisoxazole;
5-(N-tert-butoxycarbonyl-O-benzyl-L-threonin-amidomethyl)-3-bromo-4,5-dihydroisoxazole;
5-(N-benzyloxycarbonyl-L-threoninamidomethyl)-3bromo-4,5-dihydroisoxazole;
5-(N-benzyloxyzarbonyl-L-phenylalaninyl-L-alanin-amidomethyl)-3-bromo-4,5-dihydroisoxazole;
5-(N-benzyloxycarbonyl-L-alanyl-L-phenylalanin-amidomethyl)-3-bromo-4,5-dihydroisoxazole;
5-(N-benzoyl-L-phenylalaninamidomethyl)-3-bromo4,5-dihydroisoxazole;
5-(N-benzyloxycarbonyl-D-phenylalaninamidomethyl)-3-bromo-4,5-dihydroisoxazole;
5-(N-benzyloxycarbonyl-L-naphthylmethylglycin amidomethyl)-3-bromo-4,5-dihydroisoxazole;
5-(N-benzyloxycarbonyl-L-y-glutamine amidomethyl)-3-bromo-4,5-dihydroisoxazole;
5-(N-phthaloyl-L-phenylalaninamidomethyl)-3-bromo@,5-dihydroisoxazole;
5-(N-benzyloxycarbonyl-D,L-meta-tyrosinamidomethyl)-̂-9romo-4,5-dihydroisoxazole;
5-(N-benzyloxycarbonyl-L-para-tyrosinamidomethyl) 3-chloro-4,5-dihydroisoxazole; <RTI 5-(N-benzyloxyzarbonyl-L-ortho-tyrosinamidomethyl)-3-chloro-4,5-dihydroisoxazole;
5-(N-benzyloxycarbonyl-L-meta-tyrosinamidomethyl)-3-bromo-4,5-dihydroisoxazole;
5-(N-benzyloxycarbonyl-L-meta-tyrosinamidomethyl)-3-chloro-4,5-dihydroisoxazole;
5-(N-benzyloxycarbonyl-L-3-methoxyphenylalaninamido methyl)-3-bromo-4,5-dihydroisoxazole;
5-(N-benzyloxycarbonyl-L-3-methoxyphenylalaninamido-methyl)-3-chloro-4,5-dihydroisoxazole;
5-(N-benzyloxycarbonyl-L-tryptophanamidomethyl)-3-bromo-4,5-dihydroisoxazole;
5-(N-benzyloxycarbonyl-L-tryptophanamidomethyl)-3-chloro-4,5-dihydroisoxazole;
5-(N-benzyloxycarbonyl-L-5-hydroxytryptophanamidomethyl)-3-bromo-4,5-dihydroisoxazole;
5-(N-benzyloxycarbonyl-L-5-hydroxytryptophanamido-methyl)-3-chloro-4,5-dihydroisoxazole;
5-(N-benzyloxycarbonyl-L-histidinamidomethyl-3-bromo4,5-dihydroisoxazole;
5-(N-im-benzoyl-N-a-benzyloxycarbonyl-L-histidinamidomethyl)-3-bromo-4,5-dihydroisoxazole;
5-(N-toluenesulfonyl glycinamidomethyl)-3-bromo4,5-dihydroisoxazole;
5-[N-(4-benzylcarbamoyl-L-phenylalanin-amidomethyl]-3-bromo-4,5-dihydroisoxazole;
5-EN-benzyloxycarbonyl-4-(R)-hydroxy-L-prolin-amidomethyl]-3-bromo-4,5-dihydroisoxazole;
5-(N-benzyloxycarbonyl-L-para-methoxy-L-phenyl alaninamidomethyl)-3-bromo-4,5-dihydroisoxazole;
5-(Nα,0-Dibenzyloxycarbonyl-L-5-hydroxy-tryptophanamidomethyl)-3-bromo-4,5-dihydroisoxazole;
5-(N-Benzyloxycarbony 1-L-tryptophanamidomethyl)-3-bromo-4,5-dihydroisoxazole;
5(N,O,O-Tribenzyloxycarbonyl-(+)-3,4-dihydroxy-phenylalaninamidomethyl]-3-bromo-4,5-dihydroisoxazole;
5-EN-Benzyloxycarbonyl-(+)-3,4-dihydroxyphenylalaninamidomethyl]-3-bromo-4,5-dihydroisoxazole;
5-(N-Benzyloxycarbonyl-L-5-hydroxy-tryptophanamidomethyl)-3-bromo-4,5-dihydroisoxazole;
5-EN-Benzyloxycarbonyl-(+)-para-fluorophenylalanin-amidomethyl]-3-bromo-4,5-dihydroisoxazole;
5-(N-tert-butoxycarbonyl-L-phenylalanyl-L-tyrosin-amidomethyl)-3-bromo-4,5-dihydroisoxazole;
5-(Nα,N-Dibenzyloxycarbonyl-L-4-aminophenyl-alaninamidomethyl)-3-bromo-4,5-dihydroisoxazole;
5-(Nα-Benzyloxycarbonyl-Nin-formyl-L-tryptophanamidomethyl)-3-bromo-4,5-dihydroisoxazole;
5-(N-Benzyloxycarbonyl-para-amino-phenylalanlnamidomethyl)-3-bromo-4,5-dihydroisoxazole;
5-(L-Phenylalanyl-L-tyrosinamidomethyl)-3-bromo4,5-dihydroisoxazole-para-toluene-sulfonic acid;
5-(N-Benzyloxycarbonyl-5-benzyl-L-cysteinamido-methyl)-3-bromo-4,5-dihydroisoxazole;
5-(N-Benzyloxycarbonyl-L-methioninamidomethyl)-3-bromo-4,5-fl hydroisoxazole;
5-(N-enzyloxycarbonyl-O-acetyl-L-tyrosinamidomethyl)-5-(S)-3-bromo-4,5-dihydroisoxazole;
5-(N,O-Dibenzyloxycarbonyl-3-methoxy-L-tyrosinamidomethyl)-3-bromo-4,5-dihydroisoxazole;
5-(N-Benzyloxycarbonyl-3-methoxy-L-tyrosinamido-ethyl)-3-bromo-4,5-dihydroisoxazole;
5-(N-Benzyloxyzarbonyl-(+)-para-iodophenylalaninamido-methyl)-3-bromo-4,5-dihydroisoxazole; <RTI
5-[N-2-(S)-(6-methoxy-2-naphthyl)-propanoyl-L-tyrosinamidomethyl-5-(S)-3-bromo-4,5-dihydroisoxazole;
5-(N-a-benzyloxycarbonyl-L-glutamic acid-a-amidomethyl)-3-chloro-4,5-dihydroisoxazole;
5-(N-para-methoxybenzyloxycarbonyl-L-tyrosinamido-methyl)-5-(S)-3-chloro-4,5-dihydroisoxazole;
5-LN-[2-CS)-(6-methoxy-2 naphthyl)-propanoyl)-L-tyrosinamidomethyl) 5-(S)-3-chloro-4,5-dihydroisoxazole;
5-[N-(2-naphthyl-acetyl)-L-tyrosinamidomethyl-5-(S)-3-chloro-4.5-dihydroisoxazole;
5-[N-(1-Naphthyl-acetyl)-L-tyrosinamidomethyl)-5-(S)-3-chloro-4,5-dihydroisoxazole;
5-(N-isobutyloxycarbonyl-L-phenylalanin-amidomethyl)-3-bromo-4,5-dihydroisoxazole;
5-(N-succinyl-L-phenylalaninamidomethyl) <RTI 3-bromo-4,5-dihydroisoxazole;
5-(N-benzyloxycarbonyl-L-threonyl-L-phenylalaninamido-methyl)-3-bromo-4,5-dihydroisoxazole;
5-[N-cinnamoyl-L-phenylalaninamidomethyl)-3-bromo-4,-5-dihydroisoxazole
5-[N-(2(S)-6-methoxy-2-naphthylpropanoyl)-L-phenylalaninamidomethyl)-3-bromo-4,5-dihydroisoxazole;
5-EN-(2(S)-6-methoxy-2-naphthylpropanoyl)-L-phenylalaninamidomethyl]-5-(R)-3-bromo-4,5-dihydroisoxazole;
5-(N-(2-(S)-6-methoxy-2-naphthylpropanoyl)-L-phenylalaninamidomethyl]-5-(S)-3-bromo-4,5-dihydroisoxazole;
5-(N-adamantyloxycarbonyl-L-phenylalaninamidomethyl)-3-bromo-4,5-dihydroisoxazole; 5-(N-2-chlorobenzyloxycarbonyl-L-phenylalaninamido-methyl)-3-bromo-4,5-dihydroisoxazole;
5-L-(4-methoxybenzyloxycarbonyl)-L-phenyl-alaninamidomethyl]-3-bromo-4,5-dihydroisoxazole;
5-[N-(4-methoxybenzyloxycarbonyl)-L-phenyl-alaninamidomethyl]-5-(R)-3-bromo-4,5-dihydroisoxazole;
5-[N-(4-methoxybenzyloxycarbonyl)-L-phenyl-alaninamidomethyl]-5-(S)-3-bromo-4,5-dihydroisoxazole;
5-(N-tertbutoxycarbonyl-glycyl-L-phenylalaninamido-methyl)-3-bromo-4,5-dihydroisoxazole;
5-(N-a-benzyloxycarbonyl-L-lysinamidomethyl)-3-bromo-4,5-dihydroisoxazole oxalic acid;
5-(glycyl-L-phenylalaninamidomethyl)-3-bromo-4,5dihydroisoxazole oxalate salt; and 5-(N-tert-butoxycarbonyl-amidomethyl)-3-ethylsulfonyl-4,5-dihydroisoxazole.

With respect to (g) above, preferred TGase inhibitors are small molecule inhibitors comprising a 3-halo-4,5-dihydroisoxazole derivative. The TGase inhibitor may comprise an analog of isatin (2,3 diketoindoline).

Compounds for inhibition of TGases include those having the general formulae:

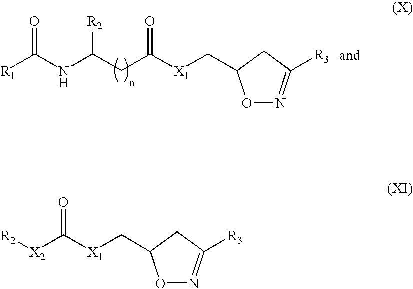

where R₁, R₂ and R₃ are independently selected from H, alkyl, alkenyl, cycloalkyl, aryl, heteroalkyl, heteroaryl, alkoxy, alkylthio, arakyl, aralkenyl, halo, haloalkyl, haloalkoxy, heterocyclyl, and heterocyclylalkyl groups. R₁ and R₂ can also be an amino acid, a peptide, a peptidomimetic, or a peptidic protecting groups.

Illustrative R₁ groups include Cbz, Fmoc, and Boc. In other embodiments of the invention, R₁ is an arylether, aryl, alkylether or alkyl group, e.g. O-benzyl, benzyl, methyl or ethyl.

R₂ groups of interest include OMe, OtBu, Gly, and Gly-NH2. In other embodiments, R₂ is selected from the group consisting of (s)-Bn, (s)-CO₂Me, (s)-Me, (R)—Bn, (S)—CH₂CONHBn, (S)— (1H-inol-yl)-methyl, and (S)—(4-hydrohy-phenyl)-methyl.

R₃ is preferably a halo group, i.e. F, Cl, Br, and I. In some embodiments of the invention R₃ is a halogen other than Cl, i.e. selected from the group consisting of I, F and Br.

X₁ and X₂ are selected from the group consisting of NH, O, and NR₄, where R₄ is a lower alkyl.

n is a whole number between 0 and 10, usually between 0 and 5, and more usually between 0 and 3.

These TGase inhibitory compounds can be readily prepared using methods known in the art, see in particular WO 2005/049064 (in particular the examples). For example, Castelhano et al have demonstrated that the dihydroisoxazole derivative(S)-1-[(3-Bromo-4,5-dihydro-isoxazol-5-ylmethyl)-carbamoyl]-2-phenyl-ethyl}-carbamic acid benzyl ester is an inhibitor of bovine epidermal transglutaminase (Castelhano et al., Bioorg. Chem. (1988) 16, 335-340).

Another example of TGase inhibitors are analogs of the dioxoindoline isatin.

Isatin analogs which may be utilised in the present invention comprise the following general formula (XII):

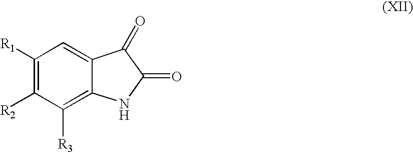

where R₁, R₂ and R₃ are may be the same or different, and are independently selected from H, a halo group, i.e. F, Cl, Br, and I, alkyls, including lower alkyls, aryls, and NO₂.

Certain specific compounds which are useful as TGase inhibitors are set forth below

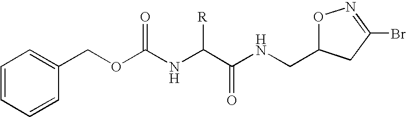

wherein R=CH₂C₆H₅, CH₂C₆H₄-p-OH, CH₂C₆H₄-p-F, CH₂C₆H₄-m-F, CH₂-3-indolyl, CH₂-3-(5-OH-indolyl), CH₂CONHCH₂Ph, (R) —CH₂Ph, or CH₃.

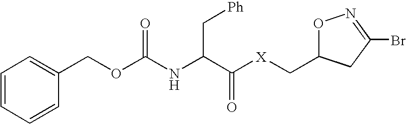

wherein X=O or NCH₃.

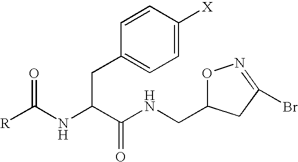

wherein X=H, R=Me or
X=OH, R=0-4-picolyl, 0-3-picolyl, OCH₂CH₂Ph, 0-2-naphthyl or

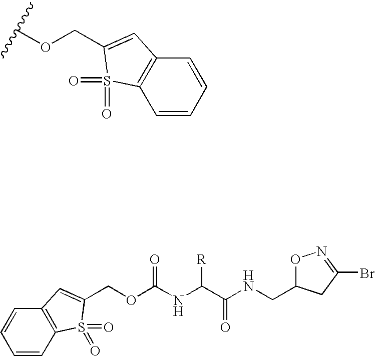

wherein R=CH₂-3-(5-OH-indolyl)

As used when describing the TGase inhibitors under heading (g), “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by hydroxy, alkoxy, aryloxy, haloalkoxy, cyano, nitro, mercapto, alkylthio, —N (R₈)₂, —C(O)OR₈, —C(O)N(R₈)₂ or —N(R₈)C(O)R₈ where each R₈ is independently hydrogen, alkyl, alkenyl, cycloalkyl, cycloalkylalkyl, aralkyl or aryl. Unless stated otherwise specifically in the specification, it is understood that for radicals, as defined below, that contain a substituted alkyl group that the substitution can occur on any carbon of the alkyl group.

“Alkoxy” refers to a radical of the formula —ORₐ where Rₐ is an alkyl radical as defined above, e.g., methoxy, ethoxy, n-propoxy, 1-methylethoxy (iso-propoxy), n-butoxy, n-pentoxy, 1,1-dimethylethoxy (t-butoxy), and the like. Unless stated otherwise specifically in the specification, it is understood that for radicals, as defined below, that contain a substituted alkoxy group that the substitution can occur on any carbon of the alkoxy group.

The alkyl radical in the alkoxy radical may be optionally substituted as described above.

“Alkylthio” refers to a radical of the formula —SRₐ where Rₐ is an alkyl radical as defined above, e.g., methylthio, ethylthio, n-propylthio, 1-methylethylthio (iso-propylthio), n-butylthio, n-pentylthio, 1,1-dimethylethylthio (t-butylthio), and the like. Unless stated otherwise specifically in the specification, it is understood that for radicals, as defined below, that contain a substituted alkylthio group that the substitution can occur on any carbon of the alkylthio group. The alkyl radical in the alkylthio radical may be optionally substituted as described above.

“Alkenyl” refers to a straight or branched hydrocarbon chain radical consisting solely of carbon and hydrogen atoms, containing at least one double bond, having from two to eight carbon atoms, and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by hydroxy, alkoxy, haloalkoxy, cyano, nitro, mercapto, alkylthio, cycloalkyl, —N(R_{8 2}, —C(O)OR₈, —C(O)N(R₈)₂ or —N(R₈)—C(O)—R₈ where each R₈ is independently hydrogen, alkyl, alkenyl, cycloalkyl, cycloalkylalkyl, aralkyl or aryl. Unless stated otherwise specifically in the specification, it is understood that for radicals, as defined below, that contain a substituted alkenyl group that the substitution can occur on any carbon of the alkenyl group.

“Aryl” refers to a phenyl or naphthyl radical. Unless stated otherwise specifically in the specification, the term “aryl” or the prefix “ar-1” (such as in “aralkyl”) is meant to include aryl radicals optionally substituted by one or more substituents selected from the group consisting of hydroxy, alkoxy, aryloxy, haloalkoxy, cyano, nitro, mercapto, alkylthio, cycloalkyl, —N(R₈)₂, —C(O)OR₈, —C(O)N(R₈)₂ or —N(R₈)C(O)R₈ where each R₈ is independently hydrogen, alkyl, alkenyl, cycloalkyl, cycloalkylalkyl, aralkyl or aryl. The term “aryl” also refers to the compound C₆H₅, i.e. Bn.

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkenyl” refers to a radical of the formula —R_{c}R_{b} where R_{c} is an alkenyl radical as defined above and R_{b} is one or more aryl radicals as defined above, e.g., 3-phenylprop-1-enyl, and the like. The aryl radical(s) and the alkenyl radical may be optionally substituted as described above.

“Alkylene chain” refers to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of aryl, halo, hydroxy, alkoxy, haloalkoxy, cyano, nitro, mercapto, alkylthio, cycloalkyl, —N(R₈)₂, —C (O)OR₈, —C(O)N(R₈) or —N(R₈)C(O)R₈ where each R₈ is independently hydrogen, alkyl, alkenyl, cycloalkyl, cycloalkylalkyl, aralkyl or aryl. The alkylene chain may be attached to the rest of the molecule through any two carbons within the chain.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of aryl, halo, hydroxy, alkoxy, haloalkoxy, cyano, nitro, mercapto, alkylthio, cycloalkyl, —N(R₈)₂, —C(O)OR₈, —C(O)N(R₈)₂ or —N(R₈)C(O)R₈ where each R₈ is independently hydrogen, alkyl, alkenyl, cycloalkyl, cycloalkylalkyl, aralkyl or aryl. The alkenylene chain may be attached to the rest of the molecule through any two carbons within the chain.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R₈)₂, —C(O)OR₈, —C(O)N(R₈)₂ or —N(R₈)C(O)R₈ where each R₈ is independently hydrogen, alkyl, alkenyl, -cycloalkyl, cycloalkylalkyl, aralkyl or aryl.

“Cycloalkylalkyl” refers to a radical of the formula —RₐR_{d} where Rₐ is an alkyl radical as defined above and R_{d} is a cycloalkyl radical as defined above. The alkyl radical and the cycloalkyl radical may be optionally substituted as defined above.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkoxy” refers to a radical of the formula —OR_{c}, where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Heterocyclyl” refers to a stable 3- to 15-membered ring radical which consists of carbon atoms and from one to five heteroatoms selected from the group consisting of nitrogen, oxygen and sulfur. For purposes of this invention, the heterocyclyl radical may be a monocyclic, bicyclic or tricyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be aromatic or partially or fully saturated. The heterocyclyl radical may not be attached to the rest of the molecule at any heteroatom atom. Examples of such heterocyclyl radicals include, but are not limited to, azepinyl, acridinyl, benzimidazolyl, benzthiazolyl, benzothiadiazolyl, benzoxazolyl, benzodioxolyl, benzodioxinyl, benzopyranyl, benzopyranonyl, benzofuranyl, benzofuranonyl, benzothienyl (benzothiophenyl), benzotriazolyl, carbazolyl, cinnolinyl, decahydroisoquinolyl, dioxolanyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thienyl, thiamorpholinyl, thiamorpholinyl sulfoxide, and thiamorpholinyl sulfone. Unless stated otherwise specifically in the specification, the term “heterocyclyll” is meant to include heterocyclyl radicals as defined above which are optionally substituted by one or more substituents selected from the group consisting of alkyl, halo, nitro, cyano, haloalkyl, haloalkoxy, aryl, heterocyclyl, heterocyclylalkyl, —OR₈, —R—OR₈, —C(O)OR₈, —R₇—C(O)ORₐ, —C(O)N(R₈)₂, —N(R₈)₂, —R₇—N(R₈)₂, and —N(R₈)C(O)R₈ wherein each R₇ is a straight or branched alkylene or alkenylene chain and each R₈ is independently hydrogen, alkyl, alkenyl, cycloalkyl, cycloalkylalkyl, aralkyl or aryl.

“Heterocyclylalkyl” refers to a radical of the formula —RₐRₑ where Rₐ is an alkyl radical as defined above and Rₑ is a heterocyclyl radical as defined above, and if the heterocyclyl is a nitrogen-containing heterocyclyl, the heterocyclyl may be attached to the alkyl radical at the nitrogen atom. The heterocyclyl radical may be optionally substituted as defined above.

In the formulas provided herein, molecular variations are included, which may be based on isosteric replacement. “Isosteric replacement” refers to the concept of modifying chemicals through the replacement of single atoms or entire functional groups with alternatives that have similar size, shape and electro-magnetic properties, e.g. 0 is the isosteric replacement of S, N, COOH is the isosteric replacement of tetrazol, F is the isosteric replacement of H, sulfonate is the isosteric replacement of phosphate etc.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminium salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The TGase inhibitors, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The device may be any medical device used internally in a subject. The introduction of the device may be by implantation or insertion for example. The device may be inside the subject for any length of time, dependent upon the particular device that is being utilised. In specific embodiments, the device is any of a catheter, stent, guidewire, sensor, ventricular assist device (VAD), graft, valve such as an aortic valve, pacemaker, artificial joint, and infusion system/pump.

In one preferred embodiment, the device is a venous catheter, preferably a central venous catheter. In an alternative embodiment, the device is a replacement joint, preferably an artificial hip or elbow joint.

In another embodiment, the coating is a hydrophilic coating. Preferably, the coating provides the device with a low friction coefficient to aid introduction of the device into a subject.

Preferably, the coating comprises a factor XIIIa inhibitor-containing polymer. The inhibitor may be covalently bound to the polymer, which may be linear or branched or a mixture thereof. The TGase inhibitor(s) may also be non-covalently incorporated into the coatings of the invention.

The inhibitor may also be reversibly bound to the polymer. By this is meant that the inhibitor may be released, at a suitable rate, from the polymer coating when in use inside the subject. For example the action of enzymes in the bloodstream of a subject, such as esterases for example, may cleave the transglutaminase inhibitor from the polymer (see below for further details).

In one embodiment, the transglutaminase inhibitor is adsorbed onto a suitable coating, such as a known hydromer coating for example. Any suitable (biocompatible) coating for a medical device may be utilised. The incorporation of one or more transglutaminase inhibitors into a stable coating facilitates a prolonged, controlled release of the inhibitor from the surface on dissolution of the biodegradable polymers.

In one embodiment, a co-polymer blend is utilised. Examples of suitable polymers into which the TGase inhibitors may be (non-covalently) incorporated include, but are not limited to, poly(hexano-6-lactone), poly(ethylene-co-vinyl acetate) (EVA), poly(ethylene oxide) (PEO), polyvinylpyrollidone, poly(tetrafluoroe-thylene) (PTFE), poly(dimethylsilo-xane), polypropylene, poly(ethyleneterephthalate) (PET), polyamides (nylons), poly(ether urethane) (e.g. Pellethane), poly(ether urethane urea) (e.g. Biomer), low density polyethylene (LDPE), high density polyethylene (HDPE), polysulfones, polyvinylchloride (PVC), poly(2-hydroxyethylmethacrylate (PHEMA) and polylactide and blends thereof such as poly(hexano-6-lactone)/polyactide blends and EVA/PEO blends. Co-polymers such as these offer a range of drug release properties and are clinically acceptable[15], and may be readily optimized for the desired release kinetics of the inhibitors.

Polymer coatings may be prepared by standard dip-coating methods. Suitable polymer-blends may be dissolved in an appropriate solvent. Examples of a solvent which may be utilised to produce coatings according to the invention include dichloromethane and methylethylketone.

Varying amounts of the inhibitor may added to the solution comprising the polymer. A preferred amount of transglutaminase inhibitor(s) for incorporation into a coating according to the invention comprises approximately 0.1-50% w/w inhibitor, preferably approximately 1-25% w/w inhibitor, even more preferably approximately 2-10% w/w inhibitor.

In one specific embodiment, the polymer which incorporates one or more transglutaminase inhibitors which preferably comprise, consist essentially of or consist of a factor XIIIa inhibitor also incorporates polyethylene glycol (PEG) molecules/monomers therein. PEG is a well known hydrophilic molecule which has many uses in the coatings of the invention. Various forms of PEG are commercially available. Examples of suitable monomers include polyethylene glycol acrylates, including mono-methoxy triethylene glycol mono(meth)acrylate, mono-methoxy tetraethylene glycol mono(meth)acrylate and polyethylene glycol mono(meth)acrylate.

In a further embodiment of this aspect of the invention, the TGase inhibitor is covalently bound to the polymer via an ester linkage. Thus, preferably the inhibitor is one which presents a (free) carboxylic acid group which can be utilised to form the ester linkage. This carboxylic acid group is preferably one which is not involved in the action of the inhibitor on transglutaminases (in particular factor XIIIa). Thus, the carboxylic acid group is preferably neither involved in inhibitor recognition by the TGase, nor in inhibitor binding by TGase. Thus, inhibitors of formula I and/or formula II are particularly useful. Most preferably compounds 1 and/or 2 are utilised.

In a preferred embodiment, the ester linkage is formed between the inhibitor and a polyethylene glycol (PEG) molecule/monomer. Such a polymer is shown in reaction scheme 1 below. These polymers have the advantage that the inhibitor may be released in a controlled manner from the coating on the device by the action of esterases found in, and produced by, the subject.

As aforementioned, there are numerous commercially available PEG molecules. Preferably, the PEG utilised in the coatings of the invention comprises a mono-methacryloyl PEG. Numerous types of mono-methacryloyl PEGs are available which allow variation of both the PEG chain length and the nature of the substitution on the PEG carbon atom which is adjacent to the ester linkage. All suitable forms of PEG are included within the scope of the invention. It is a matter of routine for the skilled person to investigate which PEG molecules allow the optimum presentation and release of the inhibitor when in use in vivo.

In one preferred embodiment, the coating comprises, consists essentially of or consists of a polymer formed from monomers comprising one or more of the specific inhibitors described above, in particular compounds of formula I and/or II, especially compounds 1 and/or 2 and derivatives thereof. The TGase (factor XIIIa) inhibitors of formula I show considerable promise for exploitation in therapeutic areas where TGase has been implicated[12]. These agents are effective only on extracellular TGases which means they do not interfere with intracellular TGases, and show no toxicity when administered to rats for periods up to 120 days[13].

Compounds 1 and 2 (as described in the experimental section below) have IC₅₀ values for factor XIIIa of 50 μM and 10 μM, respectively[14]. These compounds and their close analogues have also been shown to inhibit stabilized fibrin clot formation. They reduce cross-linking of host proteins, principally fibrin and fibronectin, and incorporation of plasminogen activator inhibitors (PAI) within thrombi, thus increasing the natural rate of fibrinolysis.

In one specific embodiment, the coating comprises, consists essentially of or consists of a polymer comprising, consisting essentially of, or consisting of the following monomer units:
PEG, preferably PEG mono-methacrylates,
styrene; and
one or more transglutaminase inhibitors, preferably a factor XIIIa inhibitor, covalently attached to a PEG molecule.

A suitable representative polymer structure is shown as polymer 1 below. The relative amounts and location and arrangement of the monomers may be varied from the arrangement shown for polymer 1, as would be readily understood by one of skill in the art.

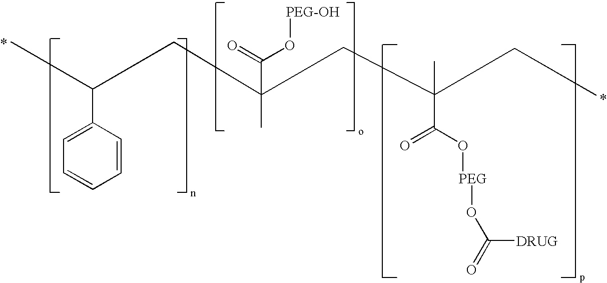

Polymer 1; wherein

“drug” represents a suitable transglutaminase inhibitor, preferably a factor XIIIa inhibitor.

n, o and p may be any appropriate number and may be the same or different for each monomer and is preferably between 1 and 1000, more preferably between 100 and 500. Preferably the styrene-derived component accounts for half of the mass of the polymer. Preferably the PEG monomer comprises, consists essentially of or consists of poly(ethylene glycol) monomethacrylate where the number of ethylene glycol units is about 10. This gives a range for the n, o and p values such that n/(o+p)=5. Having the two PEG chains present allows for variation in the drug loading whilst maintaining the overall hydrophobic-hydrophilic balance of the polymer.

Preferably, the PEG molecule attached to the inhibitor comprises mono-methacryloyl PEG, although any suitable PEG is included within the scope of the invention (e.g. as listed above).

The coating may advantageously be in the form of a hydrogel. Thus, when used in vivo, the coating will be exposed to water. The pendant PEG chains hydrate in the manner of a hydrogel but the polymer as a whole does not dissolve. Simply utilising appropriate levels of the inhibitor monomer in the polymerisation mixture may vary the levels of inhibitor to the optimum degree in order to maximally prevent thrombus formation and associated infections, whilst simultaneously ensuring there are no or minimal side effects for the subject.

### Processes for Making Suitable Polymers for the Coatings

In an additional aspect, the invention provides a process for preparing a coating as defined above (non-permanently tethered inhibitor/ester linkage) comprising esterification of a transglutaminase inhibitor (monomer), especially a factor XIIIa inhibitor with a suitable polyethylene glycol (PEG) monomer.

As discussed above, with respect to the coatings of the invention, the PEG preferably comprises mono-methacryloyl PEGs, but may be any suitable polyethylene glycol monomer, as described above.

A preferred type of esterification is Mitsunobu esterification, which is well known in the art[18].

An exemplary reaction scheme in accordance with the invention is presented as reaction scheme 2 in the experimental section below.

The process according to this aspect of the invention may further comprise polymerisation of the ester-linked transglutaminase inhibitor/PEG with additional monomers which may include for example PEG mono-methacrylates and/or styrene[18] monomers. This effectively leads to the production of a polymer which is ideally suited for use in a medical device in accordance with the invention. Further details are provided in the experimental section below.

Coatings where Inhibitor is Permanently Tethered

In an alternative embodiment, a coating for a medical device is provided wherein the transglutaminase inhibitor, which preferably comprises, consists essentially of or consists of a factor XIIIa inhibitor is permanently tethered to the polymer. By permanently tethered is meant that when the device comprising the coating is used in viva the TGase inhibitor will not, or will substantially not, be released from the coating, or will be released at a sufficiently slow rate that the device may be left inside the patient for the required period of time. Of course, the length of time the device remains inserted in the subject is dependent upon many factors, such as the nature and purpose of the device (for example catheters for drug delivery versus pacemakers, valves and artificial joint replacements), the health of the subject, the disease being treated etc.

One major advantage of this embodiment of the invention is that it helps to prevent non-specific and/or unwanted inhibition of TGases apart from those involved in fibrin stabilisation (such as factor XIIIa). Furthermore, the tethering ensures that the activity of the transglutaminase inhibitor is restricted to the locality of the device within the subject, which means that blood clotting processes elsewhere should not be adversely affected. Furthermore, tethering ensures a maximal concentration of the TGase inhibitor where it is required most, namely at the surface of the inserted medical device which are in contact with the subject.

Tethering occurs such that the inhibitor, whilst being immobilised in the coating on the device, is free to inhibit the activity of the one or more TGases, which preferably comprise, consist essentially of or consist of factor XIIIa. Thus, the portion of the inhibitor structure which binds to TGase and thus inhibits the TGase activity is unaffected by the tethering of the inhibitor to a polymer. In a preferred embodiment, the inhibitor is tethered to the polymer via an amide linkage. Such an amide linkage is stable in vivo and thus prevents release of the inhibitor from the device during use.

Preferably, the TGase inhibitor is tethered to the polymer via an amide linkage to a polyethylene glycol (PEG) molecule which forms part of the polymer. Any suitable PEG molecule may be utilised as described in more detail above, including for example any one or more of the various mono-methacryloyl PEGs.

Preferred inhibitors for inclusion in the coatings where the inhibitors are permanently tethered comprise the inhibitors described above (including those listed under headings (a) to (g)), in particular those disclosed in formula I and II, most preferably compounds 1 and 2 above and/or derivatives thereof. Molecular modelling studies have revealed that the carboxyl residue of these inhibitors remains free and accessible at the opening to factor XIIIa's active site (a specific TGase involved in clot stability), as opposed to being involved in recognition and binding of the inhibitor. Thus, the carboxyl groups may be utilised to form a stable amide linkage to tether the inhibitor to the polymer, whilst retaining TGase inhibitor activity.

The polymers themselves, as described herein are useful for forming coatings for medical devices, and thus are considered to represent a specific aspect of the invention.

### Process for Preparing Tethered Inhibitors

A process for preparing a coating comprising, consisting essentially of or consisting of a tethered transglutaminase inhibitor is also encompassed within the scope of the invention. Preferably, the TGase inhibitor comprises, consists essentially of or consists of a factor XIIIa inhibitor.

The generalised reaction scheme involves the following steps:
(a) reacting a free carboxyl group of a TGase inhibitor, with the NH₂ (amino) terminus of an oligomer comprising PEG units having a free NH₂ (amino) terminus at one end and a protected NH-terminus at the opposite end,
(b) unprotecting the protected NH-terminus of the oligomer to form a second free NH₂ terminus; and
(c) reacting this second free NH₂ terminus with methaloyl chloride to form a polymerisable monomer having a formula as presented in reaction scheme 3 below.

An exemplary reaction is provided as reaction scheme 3 in the Experimental section below.

This monomer may then be polymerised accordingly to form a coating according to the invention. Polymerisation may incorporate additional monomers to give beneficial properties in the final coating. For example, styrene monomers or oligomers formed therefrom may be reacted to form part of the polymer in any desired quantity. Similarly, monomers or oligomers of suitable PEG molecules, such as mono-methacryloyl PEGs, may be incorporated into suitable polymers for coating medical devices (for internal use).

In a further embodiment, the coatings of the invention further comprise, consist essentially of or consist of one or more antimicrobial agents. Any suitable antimicrobial agent may be utilised, which has selective toxicity for the pathogen but has no or negligible adverse effects on the subject. Any pathogen which links itself to a (forming) thrombus/blood clot may be targeted according to the present invention. As discussed above, specific target organisms include but are not limited to bacteria and yeast such as Staphylococci (in particular S. aureus and S. epidermidis) and Candida (in particular C. albicans). Of course, the subject may tolerate some side effects, but these must not be (overtly) dangerous and/or debilitating for the subject. The antimicrobial agent may have a cidal or static effect and/or may aid killing or clearance by the subject's own immune system.

The inclusion of one or more antimicrobial agents serves to kill and/or prevent growth and/or replication of pathogenic organisms released from a thrombus that is broken down as a direct consequence of the action of the one or more transglutaminase inhibitors found in the coating.

The agent may be taken from the two main types of antimicrobial agents, antibiotics (natural substances produced by microorganisms) and chemotherapeutic agents (chemically synthesized), or may be a hybrid of the two such as semi-synthetic antibiotics (a subsequently modified naturally produced antibiotic) or synthetic antibiotics (synthesised versions of natural antibiotics).

Target organisms are those organisms which become associated with a device inside the subject and cause an infection. Thus, any organism that has the potential to link itself to a blood clot may be targeted in accordance with the present invention. Suitable target organisms include those which can bind to fibrin and/or fibrinogen and/or fibronectin and other components of a blood clot. Thus, organisms which encode and express one or more fibrin and/or fibrinogen and/or fibronectin binding proteins may be targeted according to the present invention. Particularly relevant target organisms are micro-organisms such as bacteria and yeasts. For example, pathogens may become associated with devices such as heart valves and cause endocarditis and/or septic shock. In addition, aortic regurgitation may result where the infection means that a valve cannot close completely during diastole. Micro-organisms such as bacteria and yeasts can bind to proteins involved in thrombus formation on the surface of the medical device, such as fibrin/fibrinogen and fibronectin. The interaction is mediated by the production of a number of microbial surface components recognizing adhesive matrix molecules. As mentioned above, in Staphylococcus aureus these include the fibrinogen-binding clumping factors A and B and the fibronectin-binding protein (FnbA) [8]. FnbA is a substrate for TGases such as factor XIIIa and undergoes covalent cross-linking to fibrinogen and fibronectin [9,10]. Similar mechanisms involving fibronectin binding proteins and other host protein receptors apply to the other organisms involved in medical device-related infection, including Staphylococcus epidermidis and Candida albicans [10a,b].

In particular, organisms that colonise the skin of the subject are targeted, since these organisms may enter the subject at the site where the device was inserted and become associated with the device leading to masking from the immune system and causing an infection. Particularly relevant target pathogens are Gram-positive bacteria, in particular Staphylococcus and Enterococcus species. The viridans group streptococci and Streptococcus bovis are also targets implicated in infective endocarditis associated with valves, such as heart valves. A particular target is Staphylococcus aureus, as represented by strain NCTC 8325 and methicillin resistant strains which presently cause significant problems in hospital environments. Further targets are Staphylococcus epidermidis, represented by strain NCTC 11047, and yeasts such as Candida albicans, represented by strain ATCC 26555 which are known to produce fibronectin binding surface proteins [10a,b] and are capable of adhering to catheter material.

However, in one embodiment, broad-spectrum antimicrobial agents may be utilised in order to combat a number of pathogens provided there is little or no toxicity for the subject.

Suitable antimicrobial agents may have at least one or more of the following properties:
(1) the ability to prevent growth and/or replication and/or to kill pathogens which become associated with medical devices through their ability to bind to components of a thrombus and cause infections,
(2) the agent should be non-toxic to the subject and without adverse side effects,
(3) the agent should be non-allergenic to the subject,
(4) the agent should not eliminate the natural flora of the subject,
(5) the agent should be stable in the polymer and also in the coating when applied to the medical device, and when the medical device is utilised in vivo,
(6) the agent should preferably be cheap and readily available/easy to manufacture; and
(7) the agent should be sufficiently potent that pathogen resistance does not develop (to any appreciable degree).

In one embodiment, a combination of multiple suitable antimicrobial agents is utilised.

Antibiotics or derivatives thereof may be selected from the following groups; beta-lactams such as penicillin, in particular penicillin G or V, and cephalosporins such as cephalothin, semi-synthetic penicillins such as ampicillin, methicillin and amoxicillin, clavulanic acid preferably used in conjunction with a semi-synthetic penicillin preparation (such as clavamox or augmentin for example), monobactams such as aztreonam, carbapenems such as imipenem, aminoglycosides such as streptomycin, kanamycin, tobramycin and gentamicin, glycopeptides such as vancomycin and teicoplanin, lincosamides such as lincomycin and clindamycin, macrolides such as erythromycin and clarithromycin, oxazolidinones such as linezolid, streptogramins such as dalfopristin-quinupristin, polypeptides such as polymyxin and bacitracin, polyenes such as amphotericin and nystatin, rifamycins such as rifampicin, tetracyclines such as tetracycline, semi-synthetic tetracyclines such as doxycycline, chlortetracycline, chloramphenicol, quinolones such as nalidixic acid and fluoroquinolones and folate inhibitors such as sulfonamides, for example sulphamethoxazole and trimethoprim.

A most preferred antibiotic is vancomycin, since most MRSA strains have not yet acquired resistance to this antibiotic. Teicoplanin, rifampicin and tetracyclines may also be utilised.

In a still further embodiment, the coating further comprises a thrombolytic agent. The inclusion of an agent that promotes lysis of blood clots may further enhance the action of the transglutaminase inhibitor in terms of encouraging breakdown of blood clots and/or prevention of thrombus formation. Additionally, as a consequence of the release of micro-organisms such as pathogenic bacteria and yeast from blood clots the antimicrobial agent and/or host defences have improved access to the micro-organisms.

The thrombolytic agent may comprise any one or more of tPA, urokinase, retaplase, prourokinase, anisoylated purified streptokinase activator complex (APSAC) eminase and/or streptokinase. All of these and other thrombolytic agents are well known in the art and various forms are commercially available. The thrombolytic agent may be isolated from natural sources or produced by recombinant technology. Use of recombinant technology may allow variants to be produced which are considered to be included within the scope of the invention.

### Medical Devices

According to a further aspect, the invention provides a transglutaminase inhibitor immobilized on a (medical) device. Preferably, the transglutaminase inhibitor comprises, consists essentially of or consists of a factor XIIIa inhibitor.

In a still further aspect there is provided a (medical) device for introduction into a subject comprising a transglutaminase inhibitor coated on at least one surface thereof. Preferably, the transglutaminase inhibitor comprises, consists essentially of or consists of a factor XIIIa inhibitor.

The device may comprise any medical device which is introduced into a subject. The introduction of the device may be by implantation or insertion for example. The device may be inside the subject for any length of time, dependent upon the particular device which is being utilised. In specific embodiments, the device is any of a catheter, stent, guidewire, sensor, ventricular assist device (VAD), graft, valve such as an aortic valve, pacemaker, artificial joint, and infusion system/pump.

In one preferred embodiment, the device comprises, consists essentially of or consists of a venous catheter, preferably a central venous catheter.

In an alternative embodiment, the device comprises, consists essentially of or consists of a replacement joint, preferably a hip or elbow joint. Preferably, the one or more TGase inhibitors comprises, consists essentially of or consists of a factor XIIIa inhibitor.

In a preferred embodiment, all surfaces of the device exposed to the subject are coated with the inhibitor. This effectively ensures that the probability of thrombus formation induced by the inserted device is minimised. Clot stability is effectively inhibited with the direct consequence that potentially harmful micro-organisms such as pathogenic bacteria and/or yeast are released, such that they may be readily killed by antimicrobial agents and/or by the subject's immune system.

The device according to the invention may incorporate any suitable transglutaminase inhibitor. Specific inhibitors are described above. Particularly preferred inhibitors are those described in WO 2004/113363 (incorporated herein by reference) and described in detail under heading (d) above, and especially compounds of formulas I and II in particular compounds 1 and 2 and derivatives thereof.

The device may be coated with any coating of the invention as defined above. Thus, the features of the invention described with respect to the coatings of the invention apply mutatis mutandis to this aspect of the invention. In particular, a device is provided wherein the inhibitor is essentially irreversibly bound to the device (although the invention is not limited thereto; the TGase inhibitors may be covalently or non-covalently incorporated into a coating). Thus, the inhibitor is not released from the device to any significant degree during use of the device in a subject. The inhibitor is bound to the device in such a manner that it is still free to be able to act on one or more appropriate tranglutaminases, such as factor XIIIa, and thus inhibit its activity.

Alternatively, and as described above, the inhibitor may be coated on the device such that it is slowly released from the device. The inhibitor may be released at a rate such that 50% of the inhibitor remains coated on the device after a period of approximately 30 minutes, 1 hour, 2 hours, 3 hours, 4 hours, 5 hours, 6 hours, 7 hours, 12 hours, 24 hours, 2 days, 5 days, 7 days, 2 weeks, 1 month, 2 months, 1 year, 2 years, 5 years, 10 years, 15 years, 20 years or 25 years for example. The rate of release of the inhibitor may be modified according to the application of the device. For example, contrast a catheter and a pacemaker or a replacement joint, which may be present in a subject for differing amounts of time.

In a further embodiment, the stability of the inhibitor may be selected according to the desired use of the medical device. Since the inhibitors are designed to target TGases such as factor XIIIa, which are involved in clot formulation and/or stability, there is a risk of inhibiting other TGases in vivo leading to adverse consequences in the subject. This is especially the case for compounds such as L722151 (compound 3 herein), which is one example of an TGase inhibitor that has displayed enhanced thrombolysis both in vitro and in a rabbit model of femoral artery thrombosis[11]. However, compounds such as this have the ability to pass through the cell membrane (M Griffin, personal observation), leading to non-specific inactivation of intracellular transglutaminases and other potential cytotoxic effects, which may explain why inhibitor L722151 has not been progressed to the stage of clinical evaluation. Thus, it may be advantageous to ensure that the inhibitors have an appropriate half life such that they act only in the locality of the medical device in order to inhibit thrombus formation and also advantageously release pathogenic bacteria, thus rendering them more susceptible to treatment including by the subject's own immune system (e.g. by phagocytosis). This is especially relevant for inhibitors which are capable of crossing the cell membrane, in embodiments where the inhibitors are not permanently tethered to the device (e.g. through the coatings described above including an ester linkage or where the TGase inhibitors are non-covalently incorporated into the coatings) and so are released from the device over time. A half life in the orders of minutes (such as approximately 1 min, 2 mins, 5 mins, 10 mins, 20 mins, 30 mins etc), hours (such as approximately 1 hour, 2 hours, 3 hours, 5 hours, 10 hours, 20 hours etc), days (such as approximately 1 day, 2 days, 3 days, 4 days, 5 days etc), weeks (such as approximately 1 week, 2, 3, 4, 5, 10, 20 weeks etc), months (such as approximately 1 month, 2, 3, 4, 5, 6, 12, 18, 24 months, 36 months etc) or years (such as approximately 1 year, 2 years, 5 years, 10 years, 15 years, 20 years or 25 years etc) may be appropriate depending upon the application to which the device is put. Thus, a device such as an artificial joint may require more stable inhibitors, preferably tethered inhibitors, to ensure that thrombus formation is prevented over a long period of time (20+ years) whilst the device is in place in the subject.

### Producing the Devices of the Invention

In a still further aspect, the invention provides for the use of any specific coating of the invention (as described in detail above, which description applies mutatis mutandis to this aspect of the invention) for coating a medical device. The coating comprises, consists essentially of or consists of a suitable tranglutaminase inhibitor and thus serves to achieve the goal of the invention, to prevent thrombus formation in the device and to simultaneously promote release of pathogenic micro-organism such as bacteria and yeast which may have bound to the forming thrombus in order to escape immunological detection. Preferably, the TGase inhibitor comprises, consists essentially of or consists of a factor XIIIa inhibitor since this TGase is known to play an important role in clot stability.

The medical device, as stated above, is one which is for internal use in a subject. Thus, the device may be any of, by way of example and not limitation, a catheter, stent, vascular graft, guidewire, sensor, ventricular assist device (VAD), graft, valve such as an aortic valve, pacemaker, artificial joint, and infusion system/pump. Preferably, if the device comprises, consist essentially of or consists of a catheter it comprises, consist essentially of or consists of a venous catheter, in particular a central venous catheter. Preferably, if the device comprises, consists essentially of or consists of an artificial joint, it comprises, consists essentially of or consists of a hip or elbow joint (replacement joint).

In a complementary aspect of the invention, there is provided a method of producing a device of the invention comprising coating said device with a coating according to the invention (and as defined above).

The method, in one embodiment, comprises dipping the device in a suitable coating solution. Dip coating is a well known technique and suitable dipping equipment is commercially available (such as the DIPLOMAT unit, see www.diptechsystems.com). The coating solution incorporates a transglutaminase inhibitor, preferably a factor XIIIa inhibitor, as described in detail above. The coating solution is of suitable viscosity to ensure that the device is uniformly and stably coated with the coating comprising one or more transglutaminase inhibitors in order to inhibit clot stability and as a means to release micro-organisms such as pathogenic bacteria and yeast from the device.

Dip-coating may be achieved in one embodiment by immersion in aqueous solutions, followed by air drying. A range of concentrations and soaking times may be employed to determine the optimum coating conditions and loading achievable. This approach may provide short duration or longer duration protection against thrombus formation on the surface of the appropriate medical device, depending upon the coating employed. Thus, this method may be of relevance for devices such as catheters which may not be kept inside the subject for a lengthy period of time and also for devices designed to remain inside the patient indefinitely.

Dip coating may be carried out once, or multiple times prior to drying. Alternatively, intermediate drying steps may also be utilised to achieve a layer by layer build up of the coating. The coating process may be monitored by known methods, such as by measuring weight changes after each coating layer. Inhibitor entrapment within the coating may be calculated from the weight gains of the coating, since the inhibitor should be kept within the catheter coating layer[16].

Drying may occur under atmospheric conditions, or may occur under appropriate variation in temperature and/or pressure conditions.

### Methods of Use of the Coatings and Devices of the Invention

According to a specific aspect of the invention, there is provided a (combined) method of inhibiting blood clot/thrombus formation and infection caused as a result of a (medical) device introduced into a subject comprising use of a device which comprises, consists essentially of or consists of at least one surface coated with a transglutaminase inhibitor.

The invention also provides a (combined) method of inhibiting or preventing blood clot/thrombus formation and infection caused as a result of a (medical) device introduced into a subject comprising, consisting essentially of or consisting of coating at least one surface of said device with a transglutaminase inhibitor.

In an additional aspect, the invention also provides a method of inhibiting or preventing the attachment of pathologic organisms to a medical device introduced into a subject comprising, consisting essentially of or consisting of the use of a device that comprises, consists essentially of or consists of at least one surface coated with a transglutaminase inhibitor.

The invention also provides a method of inhibiting or preventing the attachment of pathologic organisms to a medical device which is to be introduced into a subject comprising, consisting essentially of or consisting of coating at least one surface of said device with a transglutaminase inhibitor.

The TGase inhibitor inhibits any TGase involved in clot formation and/or stability. Preferably, the tranglutaminase inhibitor comprises, consists essentially of or consists of a factor XIIIa inhibitor.

The infections are those caused by any micro-organism which can link itself to a blood clot and thus become associated with the medical device. Suitable target organisms include those which can bind to fibrin and/or fibrinogen and/or fibronectin and other components of a blood clot. Thus, organisms which encode and express one or more fibrin and/or fibrinogen and/or fibronectin binding proteins may be targeted according to the present invention. Specifically, the infection may be caused by pathogenic bacteria and/or yeasts such as Staphylococcus species, for example S. aureus and S. epidermidis and Candida species, such as Candida albicans. These methods encapsulate the inventive contribution of the invention. Thus, the incorporation of TGase inhibitors onto the surfaces of medical devices has multiple beneficial effects, which include preventing micro-organisms such as bacteria and yeast from covalently attaching to host proteins and increasing the rate of thrombolysis at the surface of the medical device, thus preventing dangerous thrombus formation. Release of potentially harmful micro-organisms such as bacteria and yeast during thrombolysis renders them susceptible to therapy using appropriate antimicrobial agents (as discussed above) and also to killing by the immune system. Furthermore, by targeting this step in the clotting process, the incorporation of plasminogen activator inhibitors into the clot is prevented. This effectively makes the thrombus more susceptible to degradation/lysis.

The methods are utilised in order to prevent formation of stabilised blood clots and/or associated infections associated with the introduction of a device into a subject. The methods may be used to control infections by organisms present on the skin, which can enter the subject via the site of introduction of the device. As discussed in detail above, these pathogens have the ability to bind to components of forming thrombi and thus are stably associated with the inserted device such that they are protected from clearance by the subject's immune system with adverse consequences for the subject. In one embodiment, the method is used to control gram-positive bacterial infections, in particular Staphylococcus and/or Enterococcus species. The viridans group streptococci and Streptococcus bovis are targets implicated in infective endocarditis associated with valves, such as heart valves. A particular target is Staphylococcus aureus, exemplified by strain NCTC 8325 and also methicillin resistant strains which presently cause significant problems in hospital environments. Further targets are Staphylococcus epidermidis, exemplified by strain NCTC 11047, and pathogenic yeast such as Candida albicans exemplified by strain ATCC 26555 which are known to produce fibronectin binding surface proteins and are capable of adhering to catheter material [10a].

The coating is applied to the device before it is introduced into the subject, such that the device displays improved safety and efficacy for the subject and can potentially be kept in the subject for longer periods without the need for removal and cleaning, as compared to uncoated devices (and other prior art devices). Furthermore, the method ensures that the complications typically associated with these devices such as thrombus formation and infections caused by micro-organisms such as bacteria and yeast are effectively avoided.

Preferably, all surfaces of the device are coated. The device for use in the method may be any device of the invention as described herein, which comprises, consists essentially of or consists of a suitable transglutaminase inhibitor, preferably a factor XIIIa inhibitor coated thereon. Similarly the coating for the device may comprise any of the coatings of the invention as described herein, which comprise a transglutaminase inhibitor and are useful for coating medical devices.

The methods according to these aspects of the invention may further comprise treating the subject with an antimicrobial agent particularly in embodiments where the coating does not comprise/consist essentially of an antimicrobial agent together with a tranglutaminase inhibitor. Any suitable antimicrobial agent may be utilised, which has selective toxicity for the pathogen but has negligible adverse effects on the subject. Of course, the subject may tolerate some side effects, but these must not be (overtly) dangerous and/or debilitating for the subject. The antimicrobial agent may have a cidal or static effect.

The inclusion of an antimicrobial agent serves to kill and/or prevent growth of pathogenic micro-organisms such as bacteria and/or yeast released from a thrombus that is broken down as a direct consequence of the action of the transglutaminase inhibitor found in the coating.

The agent may be taken from the two main types of antimicrobial agents, antibiotics (natural substances produced by microorganisms) and chemotherapeutic agents (chemically synthesized), or may be a hybrid of the two such as semi-synthetic antibiotics (a subsequently modified naturally produced antibiotic) or synthetic antibiotics (synthesised versions of natural antibiotics).

Broad-spectrum antimicrobial agents may be utilised in order to combat a number of pathogens provided there is little or no toxicity for the subject. Alternatively, more specific antimicrobial treatments may be directed to the preferred target pathogens described above.

Suitable properties for an antimicrobial agent are described above and apply equally to this aspect of the invention.

In one embodiment, a combination of multiple suitable antimicrobial agents is utilised.

Antibiotics or derivatives thereof may be selected from the following groups; beta-lactams such as penicillin, in particular penicillin G or V, and cephalosporins such as cephalothin, semi-synthetic penicillins such as ampicillin, methicillin and amoxicillin, clavulanic acid preferably used in conjunction with a semi-synthetic penicillin preparation (such as clavamox or augmentin for example), monobactams such as aztreonam, carbapenems such as imipenem, aminoglycosides such as streptomycin, kanamycin, tobramycin and gentamicin, glycopeptides such as vancomycin and teicoplanin, lincosamides such as lincomycin and clindamycin, macrolides such as erythromycin and clarithromycin, oxazolidinones such as linezolid, streptogramins such as dalfopristin-quinupristin, polypeptides such as polymyxin and bacitracin, polyenes such as amphotericin and nystatin, rifamycins such as rifampicin, tetracyclines such as tetracycline, semi-synthetic tetracyclines such as doxycycline, chlortetracycline, chloramphenicol, quinolones such as nalidixic acid and fluoroquinolone and folate inhibitors such as sulfonamides, for example sulphamethoxazole and trimethoprim.

A most preferred antibiotic is vancomycin, since most MRSA strains have not yet acquired resistance to this antibiotic. Teicoplanin, rifampicin and tetracyclines may also be utilised.

As described above, the coating for a device of the invention may further comprise a thrombolytic agent. The inclusion of an agent that promotes lysis of blood clots may further enhance the action of the factor XIIIa inhibitor in terms of encouraging breakdown of blood clots and/or prevention of thrombus formation in this method of the invention. Additionally, as a consequence of the release of pathogenic bacteria from blood clots the antimicrobial agent has improved access to the bacteria. The thrombolytic agent, may, in an alternative embodiment, be administered to the subject separately rather than as part of the coating on the device.

The thrombolytic agent may comprise any one or more of tPA, urokinase, retaplase, prourokinase, anisoylated purified streptokinase activator complex (APSAC) and/or streptokinase for example. All of these thrombolytic agents are well known in the art and various forms are commercially available. The thrombolytic agent may be isolated from natural sources or produced by recombinant technology. Use of recombinant technology may allow variants to be produced which are considered to be included within the scope of the invention.

### Medical Uses and Compositions

According to a still further aspect of the invention, there is provided a device for introduction into a subject, wherein the device is coated on at least one surface with a transglutaminase inhibitor, for use in surgery/therapy. Preferably, the TGase inhibitor comprises, consists essentially of or consists of a factor XIIIa inhibitor.

More specifically the invention provides a device of the invention for use in treatments such as joint replacement, valve insertion, pacemaker fitting, opening arteries and veins which may be blocked, drug delivery, sample recovery, tissue grafting etc. The device inhibits clot formation and/or infections caused by introduction of said device into a subject. The device may be any device according to the invention, as described in detail above and may be coated with any of the coatings according to the present invention. Thus, the description above applies mutatis mutandis to this aspect of the invention. Preferably, the coating is applied to all surfaces of the device which come into contact with the subject.

In a yet further aspect, the invention provides a (pharmaceutical) composition comprising a transglutaminase inhibitor and an antimicrobial agent together with a suitable carrier, diluent or excipient. Preferably, the TGase inhibitor comprises, consists essentially of or consists of a factor XIIIa inhibitor.

The novel combination of the two components in a composition is based upon the inventive insight into the link between medical device-associated thrombus formation and infections by organisms which may usually found on the skin of a subject, such as pathogenic bacteria and yeast, for example Staphylococci and Candida species, which can cause infection when a medical device (such as a catheter for example) is inserted into a subject. All target organisms referred to above may be effectively combated by the compositions of the invention, especially those which can bind to fibrin and/or fibrinogen and/or fibronectin and other components of a blood clot. Thus, organisms which encode and express one or more fibrin and/or fibrinogen and/or fibronectin binding proteins may be targeted according to the present invention.

The present invention provides the TGase inhibitors in a variety of formulations for therapeutic administration. In one aspect, the agents are formulated into pharmaceutical compositions by combination with appropriate, pharmaceutical acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the TGase inhibitors is achieved in various ways, although oral administration is a preferred route of administration. In some formulations, the TGase inhibitors are systemic after administration; in others, the inhibitor is localized by virtue of the formulation, such as the use of an, implant that acts to retain the active dose at the site of implantation.

In some pharmaceutical dosage forms, the TGase inhibitors are administered in the form of their pharmaceutical acceptable salts. In some dosage forms, the TGase inhibitor is used alone, while in others, the TGase is used in combination with other pharmaceutically active compounds.

The formulation may be targetted by any suitable means to the locality of the device which has been introduced in the subject.

Thus, the inventors have (as mentioned above) surprisingly discovered that associating TGase inhibitors with medical devices which are introduced inside a subject has the dual beneficial effects of increasing the rate of thrombolysis at the surface of the medical device and thus preventing micro-organisms such as bacteria and yeast from covalently attaching to host proteins leading to their release Release of these pathogenic micro-organisms during thrombolysis renders them susceptible to therapy using appropriate antimicrobial agents and/or the subject's immune system and thus, the compositions of the invention can provide dual functionality by first releasing pathogens from the clot, which is broken down thanks to the action of the transglutaminase inhibitor, and then directly killing or rendering harmless the pathogens.

Additionally, it will be recalled that by targeting this step in the clotting process, the incorporation of plasminogen activator inhibitors into the clot is prevented. This effectively makes the thrombus, which cannot fully form and stabilise due to the activity of the transglutaminase inhibitor, more susceptible to degradation/lysis.

The composition may be incorporated into the coatings and medical devices (for introduction into a subject) of the invention. This may be achieved by any suitable means, such as by covalent or non-covalent attachment. The description above applies mutatis mutandis here.

Preferably, the antimicrobial agent is an antibiotic or a chemotherapeutic agent, or may be a hybrid of the two such as semi-synthetic antibiotics (a subsequently modified naturally produced antibiotic) or synthetic antibiotics (synthesised versions of natural antibiotics). The antimicrobial agent may act in a prophylactic and also therapeutic manner.

Broad-spectrum antimicrobial agents may be utilised in order to combat a number of pathogens provided there is little or no toxicity for the subject. Alternatively, more specific antimicrobial treatments may be directed to the preferred target pathogens described above.

Suitable properties for an antimicrobial agent are described above and apply equally to this aspect of the invention.

In one embodiment, a combination of multiple suitable antimicrobial agents is utilised.

Antibiotics or derivatives thereof may be selected from the following groups; beta-lactams such as penicillin, in particular penicillin G or V, and cephalosporins such as cephalothin, semi-synthetic penicillins such as ampicillin, methicillin and amoxicillin, clavulanic acid preferably used in conjunction with a semi-synthetic penicillin preparation (such as clavamox or augmentin for example), monobactams such as aztreonam, carbapenems such as imipenem, aminoglycosides such as streptomycin, kanamycin, tobramycin and gentamicin, glycopeptides such as vancomycin and teicoplanin, lincosamides such as lincomycin and clindamycin, macrolides such as erythromycin and clarithromycin, oxazolidinones such as linezolid, streptogramins such as dalfopristin-quinupristin, polypeptides such as polymyxin and bacitracin, polyenes such as amphotericin and nystatin, rifamycins such as rifampicin, tetracyclines such as tetracycline, semi-synthetic tetracyclines such as doxycycline, chlortetracycline, chloramphenicol, quinolones such as nalidixic acid and fluoroquinolone and folate inhibitors such as sulfonamides, for example sulphamethoxazole and trimethoprim.

A most preferred antibiotic is vancomycin, since most MRSA strains have not yet acquired resistance to this antibiotic. Teicoplanin, rifampicin and tetracyclines may also be utilised.

The compositions of the invention may further comprise a thrombolytic agent. The inclusion of an agent that promotes lysis of blood clots may further enhance the action of the transglutaminase inhibitor in terms of encouraging breakdown of blood clots and/or prevention of thrombus formation in this method of the invention. Additionally, as a consequence of the release of pathogenic micro-organisms such as bacteria and yeasts from blood clots the antimicrobial agent has improved access to the micro-organisms.

The thrombolytic agent may comprise any one or more of tPA, urokinase, retaplase, prourokinase, anisoylated purified streptokinase activator complex (APSAC) and/or streptokinase, for example. All of these thrombolytic agents are well known in the art and various forms are commercially available. The thrombolytic agent may be isolated from natural sources or produced by recombinant technology. Use of recombinant technology may allow variants to be produced which are considered to be included within the scope of the invention.

The invention will be further defined by and understood with respect to the accompanying figures and examples in which:

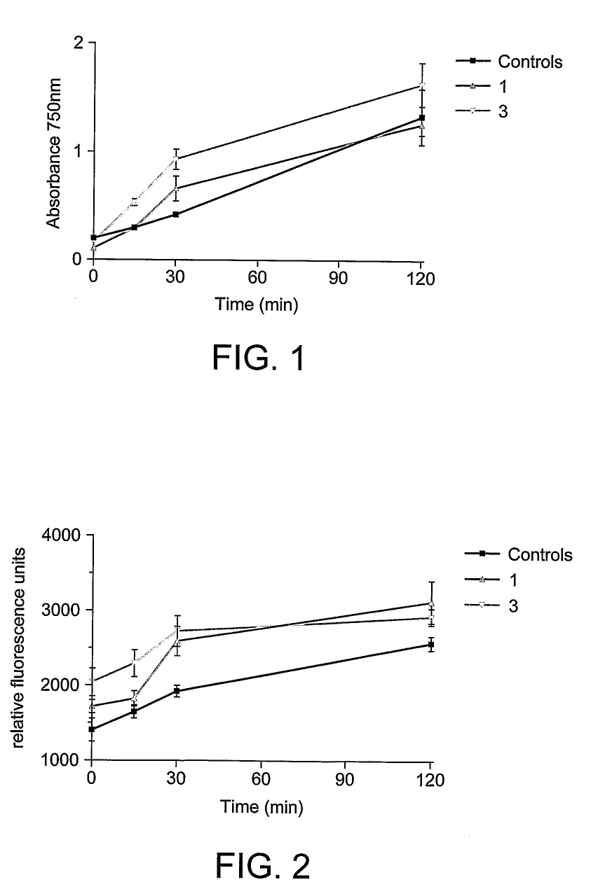

FIG. 1 shows release of red blood cells from clots formed in the presence or absence of compounds 1 and 3.

FIG. 2 shows release of FITC-fibrinogen from clots formed in the presence or absence of compounds 1 and 3.

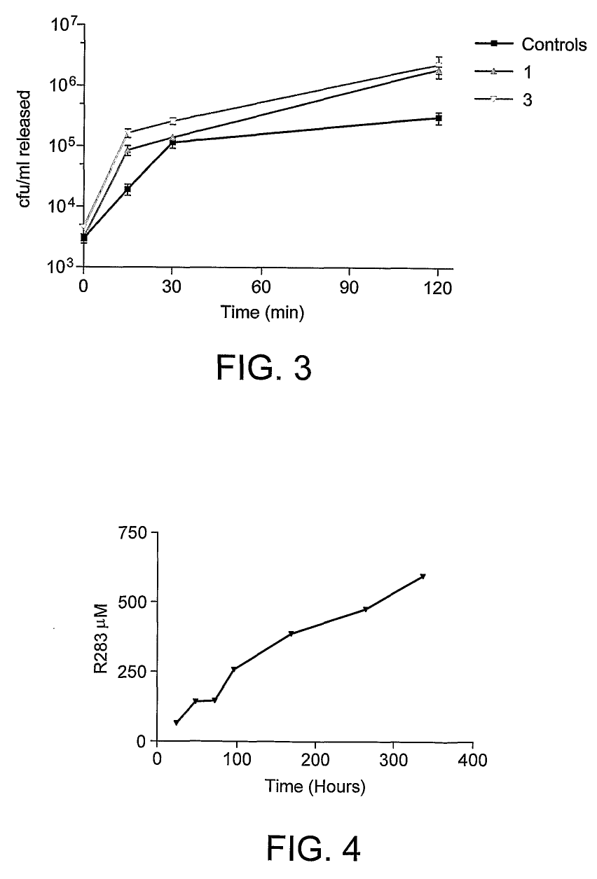

FIG. 3 shows release of Staphylococcus aureus NCTC 8325 from clots formed in the presence or absence of compounds 1 and 3.

FIG. 4 shows the release of the compound 3 inhibitor from a coated catheter over time.

### EXPERIMENTAL SECTION

Suitable quantities of inhibitors (compounds) 1 and 2 (see below) are prepared. Three coating strategies for delivery and sustained release of the inhibitors are employed together with a fourth approach involving a permanently polymer-tethered version of the inhibitors. The concepts and methodology for the preparation of the coatings are presented in detail below together with how the coatings are characterized and evaluated under physiological conditions.

### 1. Preparation of the Transglutaminase Inhibitors 1 & 2

These inhibitors are prepared according to the method of Griffin et al. [12] as shown in FIG. 1. The synthesis requires 6-diazo-5-oxo-L-norleucine (DON, which is commercially available). This is coupled to the N-hydroxysuccinimidyl active esters of N-benzyloxycarbonyl phenylalanine/proline. The diazoketone function is converted to the bromo derivative with hydrogen bromide which is displaced by dimethyl sulfide to give the final compounds in the salt form.

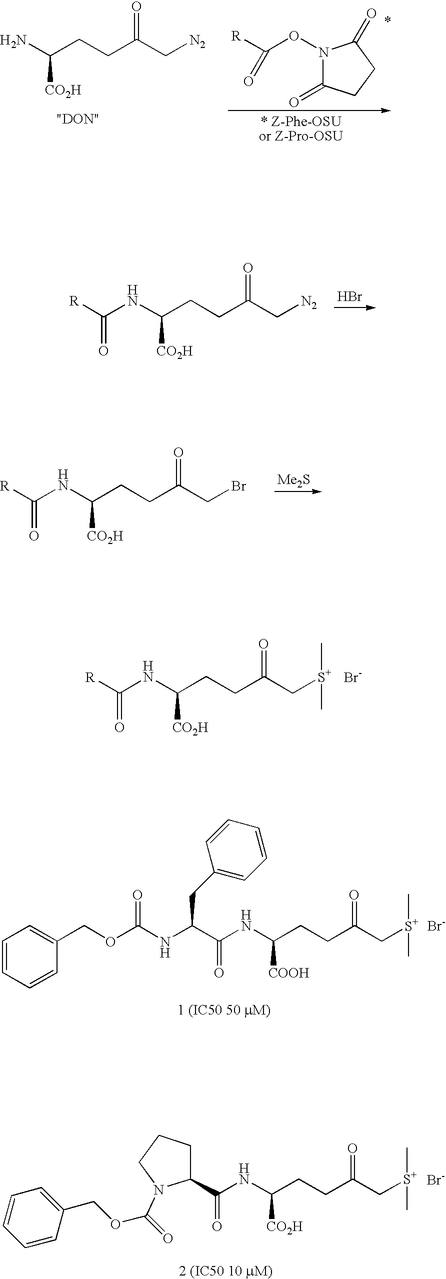

2. Simple Surface Adsorption of the Inhibitors into the Existing Hydromer Coating of Conventional Polyurethane Catheters

This approach provides short duration protection against thrombus formation on the catheter surface (e.g. 1-3 days protection). Hydromer coated polyurethane catheters are dip-coated by immersion in aqueous solutions, followed by air drying. A range of concentrations and soaking times are employed to determine the optimum coating conditions and loading achievable. Release kinetics and activity are assessed alongside the other coated preparation as described below.

3. Incorporation of the Inhibitors into Biodegradable Polylactide or Caprolactone Polymers Adsorbed onto the Catheter Surface

This approach facilitates a prolonged, controlled release of drug from the surface on dissolution of the biodegradable polymers. To achieve this, well established co-polymer blends based on e.g. poly(hexano-6-lactone) or polylactide, which offer a range of drug release properties and are clinically acceptable[15], are optimized for the desired release kinetics of the inhibitors.

Polymer coatings are prepared by the dip-coating method. Briefly, the polymer-blends are dissolved in the appropriate solvent (e.g. dichloromethane) and varying amounts (e.g. 2-10% w/w) of the inhibitor are added to the solution. Catheters and polyurethane sheets (used in subsequent testing of release kinetics) are repeatedly dip-coated in the polymer solution containing the inhibitor. The coated catheters and sheets are subsequently dried as appropriate.

The coating process is followed by measuring weight changes after each coating layer. Inhibitor entrapment within the coating is calculated from the weight gains of the coating, since the inhibitor should be kept within the catheter coating layer[16].

### 4. A Novel Biolabile Esterase-Activated Drug Release System Involving Construction of a Dual-Acting Polymer

This enables solvent casting of bio-activated polymer onto the surface of the polyurethane sheets. Upon venous insertion of a coated polyurethane catheter there is immediate hydration and acquisition of surface lubricity followed by esterase (present in biological fluids)-mediated slow release[17] of the inhibitor (as shown in the reaction scheme below).

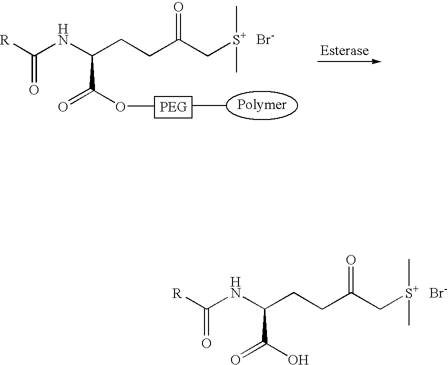

Reaction Scheme 1: Esterase-Facilitated Cleavage of Drug from Hydrogel Polymer.

The length of the chain connecting the inhibitor to the polymer backbone may be varied as well as the nature of the substitution in the chain. This affords the flexibility required to fine-tune the rate of inhibitor release. In order to prepare the polymer-linked inhibitors, polymerisable units containing the inhibitor and a polyethylene glycol (PEG) chain are prepared by Mitsunobu esterification[18] of mono-methacryloyl PEG (see the reaction scheme below). Several commercially-available mono-methacryloyl PEGs are available which allow variation of both the PEG chain length in the drug-polymer conjugate and also the nature of the substitution on the PEG carbon adjacent to the ester function.

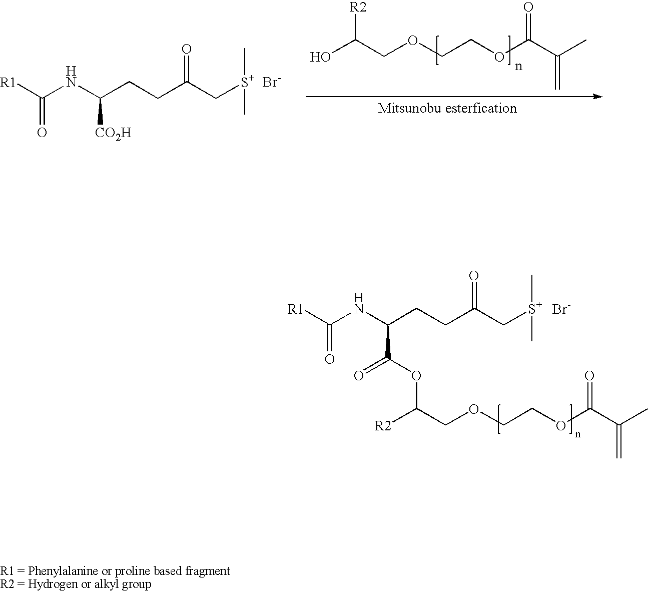

Reaction Scheme 2: Preparation of Inhibitor-Containing Polymerisable Units for Incorporation into Hydrogel-Like Coatings

These monomers may be co-polymerised with PEG mono-methacrylates diluted with styrene to give linear polymers containing the covalently-bound drugs (polymer 1). With the appropriate ratio of PEG monomer to styrene the polymers are soluble in chlorinated solvents such as dichloromethane but insoluble in water[19]. The test samples are coated with the polymer simply by dipping the sample into a solution of the polymer and allowing the solvent to evaporate. Upon contact with water the pendant PEG chains hydrate in the manner of a hydrogel but the polymer as a whole will not dissolve. With the polymerisable drug monomer in hand the loading of inhibitor in the pseudo-hydrogel matrix may be fine-tuned simply by varying the proportion of monomer present in the polymerisation mixture.

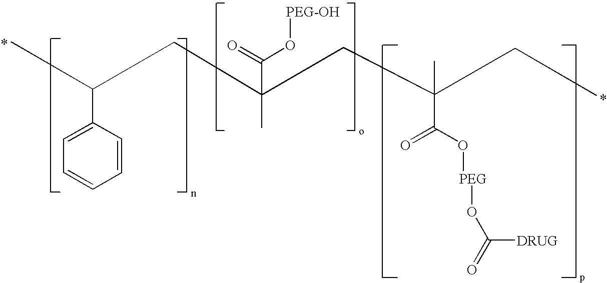

### Polymer 1: Generic Representation of Pseudo-Hydrogel Linear Polymer-Inhibitor Conjugate

### 5. Tethered Inhibitors

The inventors have conducted molecular modelling docking studies (Cache) with the drugs (inhibitors 1 and 2) and factor XIIIa which indicate that the carboxyl residue remains free and accessible at the opening to the active site rather than being involved in the drug-protein recognition and binding event. Thus the utility of the drugs whilst permanently tethered to the polymer via a polyethylene glycol using amide linkages through the carboxyl group is possible (polymer 2).

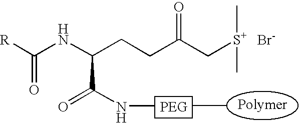

### Polymer 2: Attachment of the Inhibitor to the Peg Linker Via a Stable Amide Linkage

In this case the drug remains permanently bound to the biomedical device coating but is still able to exert its biological effect when presented with the extracellular protein. Thus factor XIIIa becomes immobilized and inactivated upon the surface of the appropriate medical device. The inhibitors (compounds) 1 & 2 are modified with commercially-available Trt-NH-(PEG)2-NH₂ under standard peptide coupling conditions (Reaction scheme 3). After removal of the trityl protecting group the exposed amine is converted to the polymerisable methacrylamide derivative. This polymerisable building block is incorporated into linear co-polymers as described in the preceding section and represented as polymer 1.

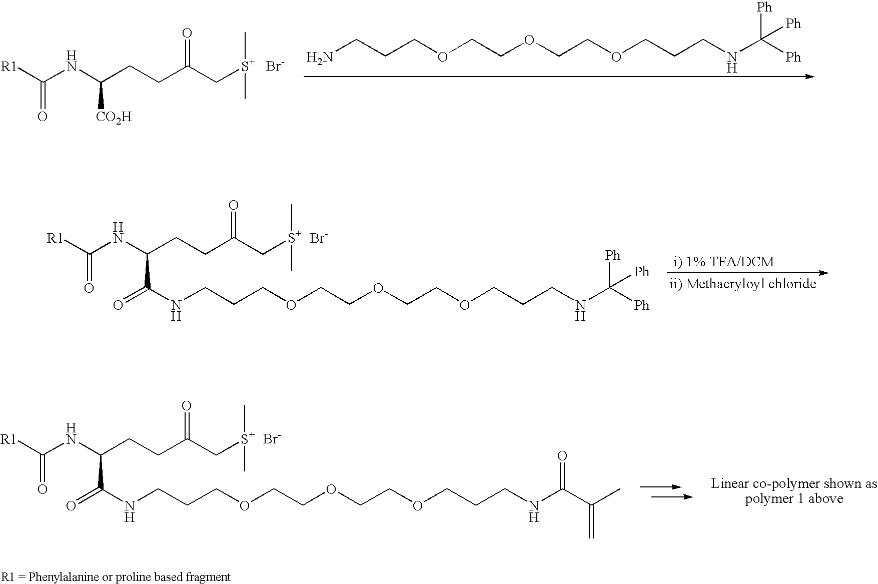

### Reaction Scheme 3: Preparation of the Required Polymerisable Amide-Linked Inhibitor-PEG Building Block

### 6. Physical Attributes of Polymer-Coated Polyurethane Catheters and Sheets

### Surface Morphology

The surface morphology of the coated catheters may be visualized using scanning electron microscopy (SEM) and Environmental SEM which allows the surface to be analysed in a hydrated condition.

### Dynamic In Vitro Release Profiles of Tgase Inhibitor Containing Catheter Coatings

Inhibitor release from coated polyurethane sheets (a standard material used to make catheters) into 0.9% NaCl at physiological temperature is measured over various time intervals (up to 6 months, depending on the type of coating) using HPLC. Coated polyurethane sheet containing various applied coatings are cut into 5 mm diameter disks to fit the wells of 96-well microtitre plates containing buffer solution. This system is used to study the kinetics of release of the inhibitors from the disks, which are made from the same material as a standard catheter. For the tethered inhibitor catheter coating requiring esterase release, commercially available esterase (porcine liver esterase, Cat. No. E3019 Sigma-Aldrich Co. Ltd.) may be used to facilitate release of inhibitor. During this time, sink conditions are maintained by continually removing and replacing fresh solvent. The effect of the various polymer blends and covalent linking of the inhibitors may be investigated. The surface morphology of the coating after various time intervals may also be investigated according to this methodology.

### Material Attributes of the Polymer Film

Briefly, polymer-TG inhibitor coated sheets are evaluated for Tensile stress-strain behaviour, flexural properties and Fatigue with the effect of drug incorporation and covalent binding all being investigated. Stress-Strain curve analyses provide a general picture of the strength and stiffness of the film and allow comparisons for polymer blend selection and give valuable information on quality control of films to ensure consistent properties during production. The stiffness of the coating may be investigated using its tensile modulus. The flexural strength (i.e. the maximum stress in the outer region of a specimen at the moment of crack or break) and also the bending stiffness of the coatings may similarly be tested as these are key characteristics for an effective (catheter) coating. Finally, in fatigue testing, a specimen of the coatings is subjected to repeated cycles of short-term stress/deformation and the coating then evaluated for the presence of micro-cracks, a decrease in toughness and tensile elongation.

### 7. In Vitro Biological Performance of Coated Polyurethane Catheters and Sheets

### Release Studies

Once the release kinetics of the inhibitors have been optimized as described above, measurement of the biological performance of the released inhibitors uses a comparable assay to that described above whereby coated polyurethane sheet containing various applied coatings are cut into 5 mm diameter disks to fit the wells of 96-well microtitre plates containing buffer solution and added factor XIIIa previously activated with thrombin and calcium. This system investigates the biological efficacy of the inhibitors once released from the catheter material disks using a suitable bioassay for inhibition of factor XIIIa, such as incorporation of [¹⁴C]-putrescine into N,N′-dimethylcasein[19] or calorimetric 96-well plate assay[21,22]. Esterase-mediated release of inhibitor from coated films may also be studied to determine the inhibition profiles in a similar manner. Controls are carried out to monitor any change in activity of factor XIIIa with time when no inhibitor is present.

### Measurement of Factor XIIIa Sequestration by Permanently-Tethered Inhibitor Co-Polymers

Coated polyurethane discs are incubated in solutions containing physiological levels of factor XIIIa and Ca²⁺ and the residual factor XIIIa activity determined using one of the bioassays (see above) to determine the amount of factor XIIIa sequestered by the polymer coating. Control assays are undertaken to monitor changes in enzyme activity with time in the absence of tethered inhibitor.

### Measurement of Fibrin Clot Formation and Lysis

The simple inhibition assays performed above allow selection of the preferred coatings which are then utilised in the next stage of biological screening. Fibrin clots are produced on polymer-coated polyurethane discs in 96-well plates by the addition of purified fibrinogen, thrombin, factor XIII and Ca²⁺. The clots are produced so as to be of a thickness equivalent to the maximum lumenal bore of a central venous catheter, for example, to mimic the required inhibitor diffusion limit expected under in vivo conditions.

The inhibition of factor XIIIa-mediated fibrin crosslinking is assessed by semi-quantitative SDS-PAGE analysis of the generated polymers at various time points. The release of Staphylococcus aureus NCTC 8325 and Staphylococcus epidermidis RP62A (strains known to produce fibronectin binding surface proteins and capable of adhering to catheter material) from the same fibrin clots is assessed by the addition of a known number of bacterial cells into the initial mixture and performing viable counting. A quantitative determination of fibrinolysis rate (to detect natural clot lysis and staphylococcal-mediated fibrinolysis) is performed by the addition of fluorescent FITC-fibrinogen into the initial mixture and monitoring of its release with time using a fluorescence microtitre plate reader[23], a technique that may be applied to fibrin, plasma and whole blood clots.

### Plasma and Whole Blood System—A Physiological System

Citrated whole human blood is taken and the separated plasma used in the same assays as for the purified fibrin clots, in order to elucidate the additional effects of inhibition of factor XIIIa-mediated crosslinking of plaminogen activator inhibitors into the polymerised fibrin. Known amounts of tissue plasminogen activator (tPA) are added in order to provide a native signal for the initiation of fibrinolysis and to optimise the fibrinolysis rate for a particular total assay time. The release rates of micro-organisms such as Staphylococcus aureus, Staphylococcus epidermidis and Candida albicans are also determined from the plasma clots, to elucidate the effects of other plasma components (e.g. fibronectin) on attachment and/or factor XIIIa-mediated crosslinking of micro-organisms to fibrin. Finally, using the information gathered from the plasma system, whole blood thrombi may be assessed in this system as a prelude to in vivo coated catheter studies.

### Proof of Principle Studies

A number of TGase inhibitors show promise for exploitation in therapeutic areas where TGase has been implicated[9,10]. Compounds 1 and 3 (below) have IC₅₀ values for factor XIIIa of 30 μM and 50 μM[10]. Compounds 1 and 3 were evaluated for stimulation of thrombolysis (human blood clot lysis) and associated release of embedded staphylococci using an in vitro assay of fibrinolysis as shown below[11].

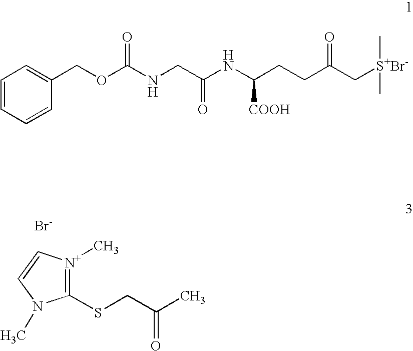

Fresh human venous blood (1 ml) was collected by venepuncture in 13 mM citrate. After addition of Staphylococcus aureus NCTC 8325 (to 10⁶ cfu/ml), tissue plasminogen activator (TPA, to 100 ng/ml) and FITC-labelled fibrinogen (to 10 μg/ml) aqueous solutions of compounds 1 or 3 (to 250 μM) or water (control), the blood was clotted by addition of CaCl₂ (to 20 mM) and allowed to cross-link for 1 hr at 37° C. Blood clots were washed three times each in 1 ml sterile phosphate buffered saline (PBS), resuspended in 1 ml PBS containing 10 μg/ml TPA and incubated with shaking at 37° C. Samples of the clot suspending fluid were withdrawn at intervals and measured for red blood cell content (absorbance 750 nm), fibrinolysis (fluorescence) and release of Staphylococcus aureus (by viable counting).

Catheter sections (2 cm, polyurethane Venflon, BD Nexiva) were immersed in a solution of compound 3 (10 mg/ml), dissolved in polylactide-glycolide in dichloromethane (256 mg/ml) and allowed to dry in air.

Individual sections were placed in 2 ml of phosphate buffered saline at 37° C. with constant shaking. Release of compound 3 was measured by assaying the phosphate buffered saline over a 336 hour period.

Results are shown in FIGS. 1 (release of red blood cells from clots formed in the presence or absence of compounds 1 and 3), 2 (release of FITC-fibrinogen from clots formed in the presence or absence of compounds 1 and 3), 3 (release of Staphylococcus aureus NCTC 8325 from clots formed in the presence or absence of compounds 1 and 3) and 4 (release of compound 3 from a polyurethane catheter). FIG. 3 clearly shows that the factor XIIIa inhibitors provide at least a 10 fold increase in release of Staphylococcus aureus NCTC 8325 from the clots as compared to the control sample. FIG. 2 similarly shows that fibrinogen release is greatly increased in the presence of factor XIIIa inhibitors, compounds 1 and 3 and FIG. 1 shows that red blood cell release from the blood clots is also increased in the presence of factor XIIIa inhibitors. FIG. 4 shows that prolonged release of a factor XIIIa inhibitor from a coated catheter can be achieved. Taken together, these results support a mechanism of action in which clot breakdown is promoted by reducing stability of the clot through inhibition of factor XIIIa.

All references referred to herein are hereby incorporated specifically as part of the present disclosure.

### REFERENCES

1. Kuter D J. 2004. The Oncologist 9:207-216
2. Slaughter S E. 2004. Postgrad Med. 116(5):59-66
3. Klerk C P, Smorenburg S M, Buller H R. 2003. Arch Intern Med. 163(16):1913-21
4. Tan K T, Lip G Y. 2005. Curr Pharm Des. 11(4):415-9
5. Petersen P. 2005. Curr Pharm Des. 11(4):527-38
6. Griffin M, Casadio R, Bergamini C M. 2004. Biochem J. Reviews 368:377-396
7. Ariens R As, Lai T-S, Weisel J W, Greenberg C S, Grant P J. 2002. Blood 100(3):743-54
7a. Bergamini C M, Dean M, Matteucci G, Hanau S, Tanfani F, Ferrari C, Boggian M, Scatturin A. Eur J Biochem. 1999. 266(2):575-82
8. Foster T J, Hook M. 1998. Trends Microbiol. 6(12):484-8.
9. Matsuka Y V, Anderson E T, Milner-Fish T, Ooi P, Baker S. 2003. Biochemistry 42(49):14643-52
10. Anderson E T, Fletcher L, Severin A, Murphy E, Baker S M, Matsuka Y V. 2004. Biochemistry 43(37):11842-52.
10a. Williams R J, Henderson B, Sharp L J, Nair S P. 2002. Infect Immun. 70(12):6805-10.
10b. Gozalbo D, Gil-Navarro I, Azorin I, Renau-Piqueras J, Martinez J P, Gil M L. 1998. Infect Immun. 66(5):2052-9.
11. Leidy E M, Stern A M, Friedman P A, Bush L R. 1990. Thromb Res. 59(1):15-26
12. Griffin M., Coutts I G, Saint R E. 2004. WO 2004/113363
13. Baumgartner W, Golenhofen N, Weth A, Hiiragi T, Saint R, Griffin M, Drenckhahn D. 2004. Histochem Cell Biol. 122(1):17-25
14. Collighan R. 2005. Unpublished observations.
15. Uhrich K E, Cannizzaro S M, Langer R S, Shakesheff K M. 1999. Chem. Rev. 99(11): 3181-3198
16. Kim H-W, Knowles J. C., Kim H-E. 2004. Biomaterials 25(7-8); 1279-1287
17. Foroutan S M and Watson D G. 1999. Int. J. Pharmaceutics 182(1); 79-92
18. Molinier V, Fitremann J, Bouchu A and Queneau Y. 2004. Tetrahedron Asymmetry 15(11); 1753-1762
19. Chun Y., Joachim K. 1999. Biomaterials 20(3):253-264
20. Lorand L, Campbell-Wilkes L K, Cooperstein L. 1972. Anal Biochem. 50(2):623-31
21. Slaughter T F, Achyuthan K E, Lai T S, Greenberg C S. 1992. Anal Biochem. 205(1):166-171
22. Song Y C, Sheng D, Taubenfeld S M, Matsueda G R. 1994. Anal Biochem. 223(1):88-92
23. Wu J H, Diamond S L. 1995. Thromb Haemost. 74(2):711-7
24. Lorand et al., 1966, Biochem. Biophys. Res. Commun. 25, 629
25. Lorand et al, 1968, Biochemistry 7, 1214
26. Lee et al, 1985, J. Biol. Chem. 260, 14689
27. Gross & Folk, 1973, J. Biol. Chem. 248, 1301
28. Gross & Folk, 1973, J. Biol. Chem. 248, 6534
29. Gorman & Folk, 1984, J. Biol. Chem. 259, 9007
30. Gross & Folk, 1973, J. Biol. Chem. 248, 6534
31. Folk & Cole, 1966, J. Biol. Chem. 241, 5518
32. Folk & Gross, 1971, J. Biol. Chem. 246, 6683
33. Gross et al., 1975, J. Biol. Chem. 250, 7693
34. Reinhardt, 1980, Appl. Biochem. 2, 495
35. Keillor, 2001, Biorg. Med. Chem. 9, 3231
36. Keillor, 2002, Biorg. Med. Chem. 10, 355

1.-56. (canceled)

57. A coating for a medical device, which device is for introduction into a subject, comprising a transglutaminase inhibitor.

58. The coating according to claim 57 wherein the transglutaminase inhibitor comprises a factor XIIIa inhibitor.

59. The coating according to claim 57 wherein the inhibitor is covalently bound to the polymer.

60. The coating according to claim 57 wherein the inhibitor comprises a compound of the formula: 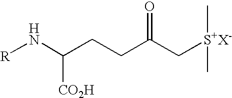
wherein R=a phenylalanine or a proline substituent and X=a pharmaceutically or veterinarily acceptable counter anion, in particular a halide counter anion and preferably bromide.

61. The coating according to claim 57 which further comprises an antimicrobial agent.

62. The coating according to claim 57 which further comprises a thrombolytic agent.

63. A transglutaminase inhibitor immobilized on a medical device, which device is for introduction into a subject.

64. A medical device for introduction into a subject comprising a transglutaminase inhibitor coated on at least one surface thereof.

65. The device according to claim 64 wherein the inhibitor comprises a compound of formula: 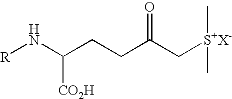
wherein R=a phenylalanine or a pro line substituent and X=a pharmaceutically acceptable counter anion, in particular a halide counter anion and preferably bromide.

66. The device according to claim 64 wherein the device is any of a catheter, stent, guidewire, sensor, ventricular assist device (VAD), graft, valve such as an aortic valve, pacemaker, artificial joint, and infusion system/pump.

67. A method of producing a device as defined in claim 64 comprising coating said device with a coating comprising a transglutaminase inhibitor.

68. A method of inhibiting blood clot formation and infection caused by pathogenic organisms which can bind to a medical device introduced into a subject comprising coating at least one surface of said device with a transglutaminase inhibitor.

69. A method of inhibiting the attachment of pathogenic organisms to a medical device introduced into a subject comprising coating at least one surface of said device with a transglutaminase inhibitor.

70. A pharmaceutical composition comprising a transglutaminase inhibitor and an antimicrobial agent together with a suitable carrier, diluent or excipient.

71. The composition according to claim 70 wherein the transglutaminase inhibitor comprises a factor XIIIa inhibitor.

72. The pharmaceutical composition according to claim 70 wherein the antimicrobial agent is an antibiotic.

73. The pharmaceutical composition according to claim 70 wherein the composition further comprises a thrombolytic agent.
